(19) United States
(12) Patent Application Publication
(10) Pub. No.: US 2010/0099155 A1
(43) Pub. Date: Apr. 22, 2010

(54) RECOVERY OF VOLATILE PRODUCTS FROM FERMENTATION BROTH

(76) Inventors: Timothy C. Frank, Midland, MI (US); Jeff A. Ferrio, Kawkawlin, MI (US); Paul E. Swanson, Indianapolis, IN (US); Juergen Lueske, Stade (DE)

Correspondence Address: The Dow Chemical Company, Intellectual Property Section, P.O. Box 1967, Midland, MI, 48641-1967, US

(21) Appl. No.: 12/516,981

(22) PCT Filed: Dec. 12, 2007
(86) PCT No.: PCT/US07/87217

§ 371 Date: May 29, 2009

Related U.S. Application Data
(60) Provisional application No. 60/875,030, filed on Dec. 15, 2006.

(51) Int. Cl. C12M 1/12; C12P 7/62; C12P 7/26; C12P 7/28; C12P 7/04; C12P 7/16; C12P 7/06

(52) U.S. Cl. 435/135; 435/148; 435/150; 435/157; 435/160; 435/161; 435/289.1; 435/297.1

(57) ABSTRACT

Provided are apparatuses and processes for the removal and purification of fermentation prepared one or more volatile organic compounds. The apparatuses comprise a fermentor unit, a vacuum side stripper unit (10), and, optionally, one or more of a pressure-swing adsorption unit, a dual-function column, a dividing wall distillation column unit, and a means for inducing phase separation of a mixture of a volatile organic compound and water.

### BACKGROUND OF THE INVENTION

(1) Field of the Invention

The invention relates to apparatuses and processes for producing volatile organic compounds (VOCs) by fermentation of a fermentation broth and removing the VOCs from the fermentation broth.

(2) Description of Related Art

Butanol is an important industrial chemical useful, for instance, as a fuel, a fuel additive, or as a precursor or intermediate in the manufacture of other useful chemicals.

The cost of producing butanol via hydroformylation technology has increased significantly in recent years due to the high cost of propylene feedstock. Production of butanol via fermentation represents an alternative process technology that utilizes a lower cost feedstock, offering the potential for lower cost of manufacture.

An important aspect of fermentation processes in the manufacture of butanol or other solvents or volatile organic compounds is the purification of the compounds. A further important aspect is the control and removal of the solvents or volatile compounds from the fermentation reactor, which, if not removed, poison the fermentation culture or reduce the culture's ability to produce the desired product, a phenomenon known as microbial inhibition. For butanol, various removal systems are known, such as pervaporation, perstraction, reverse osmosis, liquid-liquid extractions, and direct gas stripping from the fermentor vessel or reactor. However, these removal systems either do not remove enough of the volatile organic compounds or are not robust enough to be productively operated in the presence of biomass solids (e.g., cells of an organism) or under temperature and pressure conditions that are required or desired for use in commercial scale fermentation processes.

A fermentation process may employ an ultrafiltration membrane to control (typically increase) concentration of cells of an organism within a fermentor vessel, as described by Ferras, Minier, and Goma [“Acetonobutylic Fermentation: Improvement of Performances by Coupling Continuous Fermentation and Ultrafiltration,” Biotechnology and Bioengineering, vol. 28, pp. 523-533 (1986)]. The ultrafiltration membrane is used to remove liquid from cell suspensions or sludges such as those present in bio-treatment aeration basins, bioreactors, or fermentor vessels at wastewater treatment plants. Ferras et al., supra, have shown, however, that ultrafiltration membranes quickly foul when used to concentrate Clostridium acetobutylicum (ATCC 824) cultures to achieve cell concentrations of about 100 grams per liter (g/L) to 125 g/L of fermentation broth. Although a rate of fouling may be reduced by injecting bubbles of CO₂ or another gas into the fermentor vessel near the membrane's outer (fermentor-side) surface to scrub the exterior of the membrane, or by injecting a stream of liquid against the membrane surface as in a cross-flow membrane module, the rate of fouling typically remains a significant problem at high concentrations of cells in the fermentation broth. More effective uses of ultrafiltration membrane units in fermentation apparatuses and processes are needed.

Simpler and more cost effective techniques for solvent removal and/or purification than those already known, however, are needed.

### BRIEF SUMMARY OF THE INVENTION

This invention provides fermentation and stripping processes and apparatuses that efficiently and cost effectively produce by fermentation one or more VOCs (e.g., 1-butanol) in a fermentation broth (broth) in a batch, fed-batch (also known as semi-batch), or continuous fermentor vessel and strip the one or more VOCs from the fermentation broth.

A first embodiment of the present invention is a fermentation apparatus comprising a fermentor unit and a vacuum side stripper (VSS) unit;
the fermentor unit comprising a fermentor vessel, the fermentor vessel having an exterior surface and an interior surface, the fermentor vessel surfaces being spaced apart from, and generally parallel to, each other so as to define an enclosed volumetric space, the fermentor vessel having defined therein at least two apertures, a first fluid aperture and a second fluid aperture, the at least two apertures being in fluid communication with the enclosed volumetric space;
the VSS unit comprising a stripper vessel, the stripper vessel having an exterior surface and an interior surface, the stripper vessel surfaces being spaced apart from, and generally parallel to, each other so as to define an enclosed volumetric space; the stripper vessel having disposed therein two or more side-by-side stripper compartments, each stripper compartment being separated from an adjacent stripper compartment by a vertical partition member; at least a bottom portion of each vertical partition member either having defined therein a liquid trafficking aperture or being spaced apart from a bottom portion of the stripper vessel so as to define a liquid trafficking conduit between the bottom portion of the partition member and the bottom portion of the stripper vessel, or a combination thereof; at least a top portion of each vertical partition member either having defined therein a vapor trafficking aperture or being spaced apart from a top portion of the stripper vessel so as to define a vapor trafficking conduit between the top portion of the partition member and the top portion of the stripper vessel, or a combination thereof; the stripper compartments being in sequential fluid communication with each other, adjacent stripper compartments being in fluid communication with each other via a liquid trafficking conduit, a liquid trafficking aperture, or a combination thereof, and via a vapor trafficking conduit, a vapor trafficking aperture, or a combination thereof; the stripper vessel having defined therein at least four apertures, a third fluid aperture, a gas aperture, a vapor aperture, and a first agitation aperture, each of the at least four apertures being in fluid communication with the enclosed volumetric space of the stripper vessel;

the first fluid aperture of the fermentor vessel being operatively connected to the third fluid aperture of the stripper vessel to establish fluid communication between the fermentor vessel and the stripper vessel. The gas and vapor apertures in the stripper vessel preferably occur in surfaces of an upper portion of the stripper vessel. The first fluid aperture in the fermentor vessel preferably occurs in surfaces of a side portion or lower portion of the fermentor vessel; the second fluid aperture preferably occurs in surfaces of a side portion or upper portion of the fermentor vessel; and the third fluid aperture of the stripper vessel preferably occurs in surfaces of a side portion or upper portion of the stripper vessel.

A second embodiment of the present invention is a process comprising the steps of: disposing a fermentation broth in the enclosed volumetric space of a fermentor vessel of an apparatus as in the first embodiment, the broth comprising water, a plurality of cells of an organism, and a nutrients feed; allowing the nutrients feed to be fermented by the organism to produce at least one volatile organic compound (VOC); and stripping the at least one VOC from the fermentation broth. Preferably, each of the at least one VOC has a molecular weight of less than 500 grams per mole.

Additional embodiments are described in accompanying drawings and the remainder of the specification.

### BRIEF DESCRIPTION OF THE DRAWINGS

FIG. 1 depicts an illustration of an example of an agitation VSS unit, the agitation VSS unit including aeration-style mixing impellers for gas-liquid contacting.

FIG. 2 depicts an illustration of an example of a circulation VSS unit suitable for use in the invention apparatus and process in place of the agitation VSS unit of FIG. 1.

FIG. 3 shows an Aspen flow diagram from Example 1 that is used in the simulation of an ABE fermentation utilizing the fermentor unit and agitation VSS unit of the invention apparatus and process.

FIG. 4 graphically depicts, from Example 1, fit of calculated NRTL and UNIQUAC vapor-liquid equilibrium (VLE) and liquid-liquid equilibrium (LLE) data with literature VLE and LLE data for the quaternary system butanol+acetone+water+ethanol at 25° C.

### DETAILED DESCRIPTION OF THE INVENTION

The present invention provides improved apparatuses and processes for stripping and/or purifying of organic products from fermentation reactions. While the invention has been described with reference to an ABE fermentation process, the invention is also applicable to other fermentation processes, including fermentation processes for producing isopropanol and ethanol and other VOCs described herein. At any point in an invention apparatus or process for producing one or more VOCs, the one or more VOCs, being either wet or dry, and substantially pure or a mixture of two or more of the VOCs, may be sent to a separate manufacturing stream as a solvent, may be packaged for commercial sale or storage, or may be processed further as described herein. Further processing includes drying (e.g. in a pressure-swing adsorption (PSA) unit) a wet VOC or a wet mixture of two or more VOCs to produce a substantially dry VOC or a substantially dry mixture of two or more VOCs, which are useful as solvents or as a feedstock in, for example, production of a derivative thereof. Still further, the substantially dry mixture of two or more VOCs may be purified (e.g., in a dividing-wall distillation column (DWC) unit) by separating the two or more VOCs from each other to separately produce two or more substantially pure VOCs.

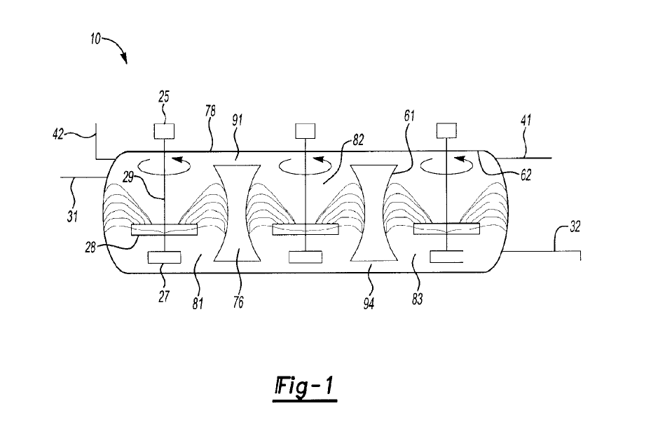

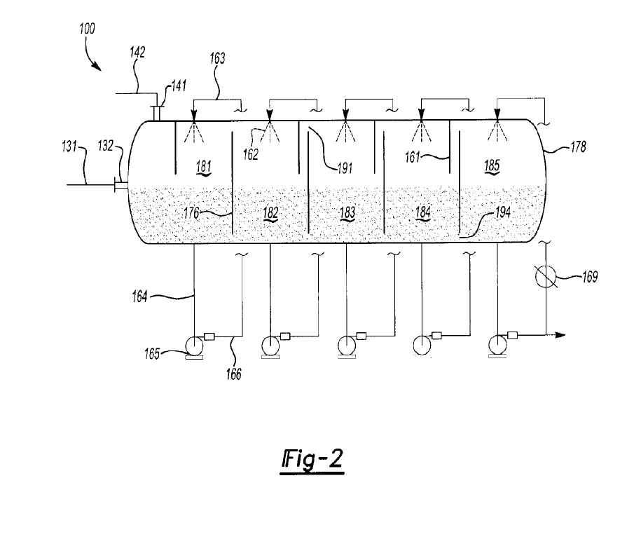

In addition to a fermentor unit 200 (not shown) and a VSS unit (e.g., 10 in FIG. 1 or 100 in FIG. 2), the apparatus and process of the present invention may further comprise one or more additional components such as, for example, an ultrafiltration membrane 822 (not shown), a membrane unit 800 (not shown), dual-function column 300 (not shown), PSA unit 400 (not shown), DWC unit 500 (not shown), or liquid-liquid extractor 700 (not shown). More additional components are described below.

A VSS unit comprises an agitation VSS unit (e.g., 10 of FIG. 1) or a circulation VSS unit (e.g., 100 of FIG. 2). An example of an agitation VSS unit 10 is depicted in FIG. 1 and described here. In FIG. 1, agitation VSS unit 10 comprises, among other components, stripper vessel 78 having an interior surface 62 and defining two fluid apertures 33 and 34, three agitation apertures 35, 36 and 37, a gas aperture 45, and a vapor aperture 46 (all not shown). A proximal stripper compartment 81, center stripper compartment 82, and distal stripper compartment 83 are disposed side-by-side within stripper vessel 78. Adjacent stripper compartments (e.g., 81 and 82; and 82 and 83) are spaced apart from each other by (I-profile) vertical partition members 76 disposed within stripper vessel 78, each vertical partition member 76 having exterior surfaces 61. Other profiles for vertical partition members are contemplated, the other profiles being, for example, I-profile.

Bottom portions 51 (not indicated) and top portions 52 (not indicated) of each vertical partition member 76 are spaced apart from bottom portions 53 (not indicated) and top portions 54 (not indicated) of stripper vessel 78 so as to define two liquid trafficking conduits 94 and two vapor trafficking conduits 91, respectively. The stripper compartments 81 to 83 are in sequential fluid communication via liquid trafficking conduits 94 and vapor trafficking conduits 91.

Two vertical partition members 76 are in fluid communication with the interior 57 (not indicated) of stripper vessel 78. Different ones of first and second impellers 28 and 27, respectively, are in fluid communication with the interior 57 (not indicated) of stripper vessel 78 and different stripper compartments (81, 82, or 83).

Certain components shown in proximity to stripper vessel 78 are three stir motors 25, three stir shafts 29, three first impellers 28, and three second impellers 27. Stripper vessel 78, stir motors 25, stir shafts 29, three first impellers 28, three second impellers 27, and vertical partition members 76 comprise an example of an agitation VSS unit. For illustration, stripper vessel 78 is shown with fermentation broth being disposed therein, but fermentation broth is not part of agitation VSS unit 10.

The three stir shafts 29 are in operative contact with stripper vessel 78 at different ones of the three agitation apertures 35, 36, 37 thereof (not indicated). The three stir shafts 29 span between and are in operative connection with different ones of the three stir motors 25 and first and second impellers 28 and 27, respectively, and are in fluid communication with a different stripper compartment (e.g., 81, 82, or 83) of stripper vessel 78.

Referring again to FIG. 1, stripper vessel 78 is in operative connection to, and fluid communication with, fluid conduits 31 and 32 at the two fluid apertures (not indicated); in operative connection to, and fluid communication with, fluid conduit 42 at the vapor aperture 46 (not shown), and in operative connection to, and fluid communication with, fluid conduit 41 at the gas aperture 45 (not indicated), the apertures being defined in stripper vessel 78.

During operation of VSS unit 10, stripper vessel 78 may receive a stream of fermentation broth, or a clarified liquid derived therefrom, from a fermentor vessel (e.g., a standard fermentor vessel 211 (not shown) or a reboiled fermentor vessel 222 (not shown)) or membrane vessel 811 (not shown) via fluid conduit 31 and through fluid aperture 33 (not indicated). Stripper vessel 78 may receive an injection of a stripper gas from a stripping gas source 11 (not shown) via fluid conduit 41 and the gas aperture 45 (not indicated). Stripper vessel 78 may send fermentation broth, or a clarified liquid derived therefrom, through a fluid aperture 34 and fluid conduit 32 back to the fermentor vessel (e.g., 211 and 222, not shown) or to waste. Stripper vessel 78 may release stripped wet VOCs through the vapor aperture 46 (not indicated) via fluid conduit 42 to an additional component such as, for example, a dual-function column 300 (not shown), a blower 350 (not shown), a first vacuum pump/compressor 370 (not shown), a vapor/liquid condenser 600 (not shown), or a PSA unit 400 (not shown).

Also during operation, stir motors 25 are actuated and cause stir shafts 29 and first and second impellers 28 and 27, respectively, to rotate, where first impeller 28 agitates fermentation broth to produce splashes (shown by way of parabolic lines between first impellers 28 and stripper vessel 78) of fermentation broth. Splashes reach exterior surfaces 61 of vertical partition members 76 and interior surfaces 62 of stripper vessel 78.

Other types VSS units that are not agitation VSS units are contemplated herein and include circulation VSS units (e.g., 100 of FIG. 2). An example of a circulation VSS unit 100 is depicted in FIG. 2.

Referring to FIG. 2, circulation VSS unit 100 comprises, among other components, stripper vessel 178, having disposed therein five stripper compartments 181, 182, 183, 184, 185. Four vertical partition members 176 are disposed within, and are spaced apart from, stripper vessel 178. Stripper vessel 178 defines one fluid aperture 132, ten agitation apertures 135 to 139 and 150 to 154 (not indicated), a gas aperture 145 (not indicated), and a vapor aperture 141. Certain components shown in proximity to stripper vessel 178 are valve 169, five liquid pumps 165, five fluid conduits 164, five fluid conduits 166, five fluid conduits 163, five nozzles 162, four (optional) splash plates 161, and four (disc-shaped) vertical partition members 176. Stripper vessel 178, liquid pumps 165, fluid conduits 164, fluid conduits 166, fluid conduits 163, nozzles 162, (optional) splash plates 161, and vertical partition members 176 comprise an example of a circulation VSS unit, 100. For illustration, stripper vessel 178 is shown with fermentation broth being disposed therein, but fermentation broth is not part of circulation VSS unit 100.

Referring again to FIG. 2, at stripper vessel 178 is in five separate and sequential operative connections, at different ones of the ten agitation apertures 135 to 139 and 150 to 154 thereof (not indicated), and is in five separate and sequential fluid communications with, fluid conduits 164, liquid pumps 165, fluid conduits 166, fluid conduits 163, and nozzles 162.

Bottom portions 172 of four vertical partition members 176 are spaced apart from bottom portions 174 of stripper vessel 178 so as to define liquid trafficking conduits 194 and top portions 173 of vertical partition members 176 are spaced apart from top portions 175 of stripper vessel 178 so as to define vapor trafficking conduits 191. The four vertical partition members 176 are also spaced apart from each other and, together with stripper vessel 178, define five stripper compartments 181 to 185, including proximal stripper compartment 181, sequentially from left-to-right three intermediary stripper compartments 182, 183, and 184, respectively, and a distal stripper compartment 185. Three four vertical partition members 176 are in fluid communication with the interior 156 (not indicated) of stripper vessel 178. The stripper compartments 181 to 185 are in sequential fluid communication with each other via the liquid trafficking conduits 194 and vapor trafficking conduits 191.

Referring again to FIG. 2, stripper vessel 178 is in operative connection to, and fluid communication with, fluid conduit 131 at fluid aperture 132 and fluid conduit 142 at vapor aperture 141. Stripper vessel 178 is also in operative connection to, and fluid communication with, a fluid conduit 133 (not shown) at gas aperture 145 (not indicated), the apertures being defined in stripper vessel 178.

During operation of VSS unit 100, stripper vessel 178 may receive a stream of fermentation broth, or a clarified liquid derived therefrom, from a fermentor vessel (e.g., a standard fermentor vessel 211 (not shown) or a reboiled fermentor vessel 222 (not shown)), or membrane vessel 811 (not shown) via fluid conduit 131 and through fluid aperture 132. Stripper vessel 178 may receive an injection of a stripper gas from a stripper gas source 111 (not shown) via a fluid conduit 133 (not shown) and through the gas aperture 145 (not shown). Stripper vessel 178 may send fermentation broth, or a clarified liquid derived therefrom, through another fluid aperture 134 (not shown) and fluid conduit 171 (not shown) back to the fermentor vessel (e.g., 211 and 222) or to waste. Stripper vessel 178 may release stripped wet VOCs through vapor aperture 141 via fluid conduit 142 to an additional component such as, for example, the additional components mentioned above in the description of FIG. 1.

The circulation VSS unit 100 comprises pumped-liquid circulation loops (164, 165, 166, 163, and 162) for gas-liquid contacting. During operation, liquid pumps 165 are actuated and cause fermentation broth to circulate sequentially through fluid conduits 164, liquid pumps 165, fluid conduits 166, and fluid conduits 163, and out nozzles 162 into stripper vessel 178. Sprays (not shown) of fermentation broth reach splash plates 161 and vertical partition members 176 and interior surfaces 155 (not indicated) of stripper vessel 178. During such a pumping operation, a level of fermentation broth preferably remains about the same.

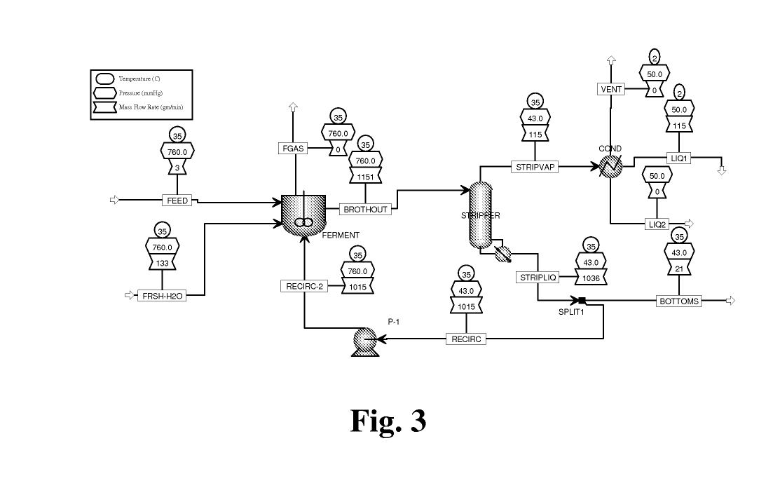

As described in detail below in Example 1 and briefly here, FIG. 3 schematically depicts, for an embodiment of an invention apparatus and process, an Aspen flow sheet illustrating conventional elements comprising, among other things, a standard fermentor unit (FERMENT), an agitation VSS unit (STRIPPER), valve (Ø), liquid pump (P-1), and vapor/liquid condenser (COND). The standard fermentor unit (FERMENT) is in sequential fluid communication with an agitation VSS unit (STRIPPER) via line BROTHOUT, valve (Ø), liquid pump (P-1), and back to standard fermentor unit (FERMENT). The agitation VSS unit (STRIPPER) is in fluid communication with the vapor/liquid condenser (COND). The standard fermentor unit (FERMENT) is also in fluid communication with a nutrients feed (FEED), a source of fresh water (FRSHH2O), and a fermentation gas vent (FGAS). See Example 1 for detailed results of the Aspen modeling.

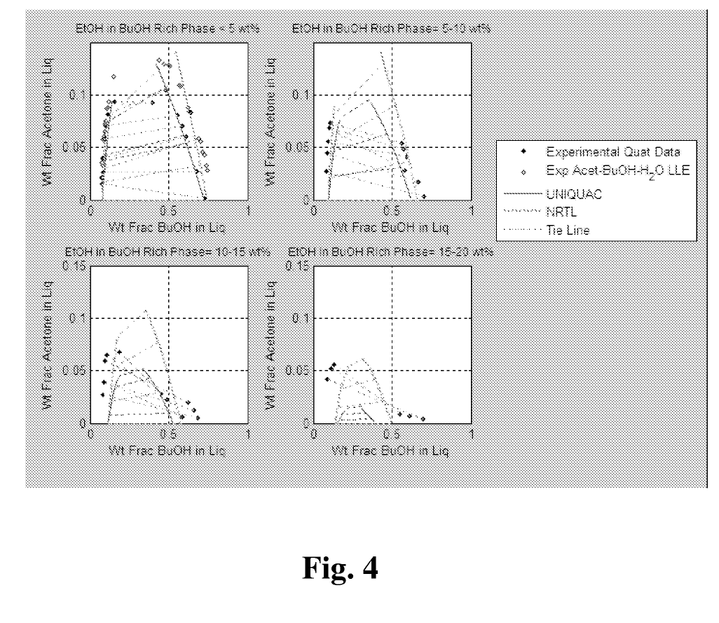

As described in detail below in Example 1 and briefly here, FIG. 4 graphically depicts examples of a fit of data from a non-random two liquid activity coefficient (NRTL) model and data from a UNIQUAC activity coefficient (UNIQUAC) phase equilibrium model to Dortmund Databank Set [2121] literature data are graphically shown in FIG. 4. Referring to FIG. 4, experimental data (i.e., Dortmund Databank Set [2121] literature data) for a quaternary system comprising acetone, ethanol, butanol, and water are plotted using a solid-diamond symbol, experimental LLE data (i.e., Dortmund Databank Set [2121] literature data) for a ternary system comprising acetone, butanol, and water are plotted using an open-diamond symbol, calculated UNIQUAC data are plotted with a solid line, calculated NRTL data are plotted with a dotted line, and dashed lines are drawn between plotted data.

A third embodiment of the invention is an apparatus for preparing organic compounds comprising: a fermentation setup comprising a fermentor vessel (e.g., a standard fermentor vessel 211 (not shown) or a reboiled fermentor vessel 222 (not shown)) and a decanter for receiving organic and aqueous components from the fermentor vessel (e.g., 211 and 222); an additional component selected from a PSA unit 400, a dual function column 300, a vacuum side stripper (e.g., 10 in FIG. 1 and 100 in FIG. 2), a DWC 500, and combinations thereof. Particularly, the apparatus comprises: a fermentor unit 200, decanter 650, and an additional component selected from the group consisting of a PSA unit 400, a dual function column 300, a VSS unit (e.g., 10 of FIG. 1 or 100 of FIG. 2), a DWC unit 500, and a combination of two or more said additional components, the fermentor unit 200 comprising a fermentor vessel (e.g., 211 and 222), the fermentor vessel (e.g., 211 and 222) being in sequential operative connection (at inlets and outlets) and fluid communication with the VSS unit (e.g., 10 of FIG. 1 or 100 of FIG. 2) and the decanter 650; or the decanter 650 and PSA unit 400 or DWC unit 500. According to the invention, at least one of the aforementioned additional components is incorporated into a fermentation apparatus. In some embodiments, two or more of the additional components may be incorporated.

Articles “a” and “an” refer to singular and plural forms of what is being modified by the articles. The term “or” refers to members in a list either singly or in any combination.

The term “comprising,” which is synonymous with the terms “including,” “containing,” “having,” “group of,” and “characterized by,” is inclusive or open-ended. These terms do not exclude additional elements, materials, ingredients, or process steps, including unrecited ones, even if the additional elements, materials, ingredients, or process steps are present in major amounts. When the term “comprising” is used as a transition from a claim's preamble to the claim's body (i.e., as a transitional term), the entire claim is open-ended (although a specific element or step within the claim may be limited by a phrase such as “consisting of” or “consisting essentially of”).

The phrases “consisting of” or “group consisting of” are closed terms. These phrases exclude any element, step, or ingredient not specified. When the phrase “consisting of” is used as a transitional phrase in a claim, the phrase closes the claim to the inclusion of materials, elements, or steps that are not specifically recited in the claim except for impurities ordinarily associated therewith and materials, elements or steps that are unrelated to the claimed invention. When the phrase “consisting of” is used in a clause of the body of the claim rather than immediately following the preamble, it limits only the element, step, or material set forth in that clause and other elements, materials, or steps outside of the clause are not excluded from the claim. The present invention also includes embodiments written by modifying the “comprising” embodiments described elsewhere herein by replacing the transitional term “comprising” with the transitional phrase “consisting of.” When used, the transitional phrase “consisting of” excludes one or more base additional components selected from the group consisting of: a PSA unit 400 (not shown), a means for inducing phase separation (e.g., liquid-liquid extractor 700, not shown), and a means for separating water from one or more VOCs (e.g., DWC unit 500, azeotropic distillation unit, and adsorbent unit, all not shown), but does not exclude one or more supplemental additional components selected from the group consisting of: blowers 350 (not shown), vacuum pump/compressors 370 and 380 (not shown), decanters 650 (not shown), vapor/liquid condensers 600 (not shown), and gas/liquid separators 660 (not shown).

The phrase “consisting essentially of” may be used in a claim's preamble to limit the scope of the claim to the specified materials, elements, or steps and those that do not materially affect the basic and novel characteristic or characteristics of the claimed invention. Referring to preambles, a “consisting essentially of” claim occupies a middle ground between closed claims that are written in “consisting of” format and fully open claims that are drafted in a “comprising” format. The present invention also includes embodiments written by modifying the “comprising” embodiments described elsewhere herein by replacing the transitional term “comprising” with the transitional phrase “consisting essentially of.” When used, the transitional phrase consisting essentially of may exclude one or more of the base and supplemental additional components mentioned above.

### Volatile Organic Compound (VOC)

A “volatile organic compound” means a molecule consisting of the elements carbon, hydrogen, and oxygen and has a molecular weight of less than 500 grams per mole. Preferably, a VOC has a molecular weight of less than 250 grams per mole. More preferably, the VOC independently is selected from the group consisting of: HO—(C₁-C₈)alkyl; HO—(C₂-C₈)alkylene-O—(C₁-C₄)alkyl; (C₃-C₈)alkanone; HO—(C₃-C₈)alkanone; (C₁-C₈)alkyl-C(O)O—(C₁-C₄)alkyl; [oxo-(C₃-C₈)alkyl]-C(O)O—(C₁-C₄)alkyl; (C₀-C₆)alkylene-[C(O)O—(C₁-C₄)alkyl]₂; O—[(C₁-C₄)alkyl]₂; and [oxo-(C₂-C₄)alkyl]-O—(C₁-C₄)alkyl.

A “(C₁-C₄)alkyl” and “(C₁-C₈)alkyl” mean an unsubstituted, branched or straight chain, saturated hydrocarbon radical of from 1 to 4 and 1 to 8 carbon atoms, respectively. A “(C₂-C₈)alkylene” and “(C₀-C₆)alkylene” mean an unsubstituted, branched or straight chain, saturated hydrocarbon diradical of from 2 to 8 and 0 to 6 carbon atoms, respectively. A (C₀)alkylene means the alkylene is absent. A “(C₃-C₈)alkanone” and “(C₂-C₄)alkanone” mean a branched or straight chain saturated hydrocarbon of from 3 to 8 carbon atoms and 2 to 4 carbon atoms, respectively, that is mono-substituted by an oxo (i.e., ═O) group on any one of the carbon atoms except terminal carbon atoms, wherein the hydrocarbon is otherwise unsubstituted. A “HO—(C₃-C₈)alkanone” is a hydroxy-substituted (C₃-C₈)alkanone, which is as defined previously. An “[oxo-(C₃-C₈)alkyl]” and “[oxo-(C₂-C₄)alkyl]” is a carbon radical of (C₃-C₈)alkanone and (C₂-C₄)alkanone, respectively, wherein (C₃-C₈)alkanone and (C₂-C₄)alkanone are as defined previously.

Preferred VOCs are acetone, ethanol, 1-butanol, 2-butanol, 2-methyl-1-propanol, 1-pentanol, 1-hexanol, 2-butoxyethanol, 1-butoxy-2-propanol, hydroxyacetone, ethyl acetate, and 2-oxo-ethyl acetate; more preferred are acetone, ethanol, 2-propanol, 1,3-propanediol, 1-butanol, and 1,4-butanediol; still more preferred is ethanol and 1-butanol. In an invention process for producing a particular VOC, where the process produces two or more VOCs, preferably the particular VOC is produced as the major component of the two or more VOCs, i.e., the particular VOC is produced at time-averaged concentration in the fermentation broth in a fermentor vessel (e.g., a standard fermentor vessel 211 (not shown) or a reboiled fermentor vessel 222 (not shown)) that is greater than the time-averaged concentration of any other VOC in the fermentation broth in the fermentor vessel (when a reboiled fermentor vessel 222 is used, the time-averaged concentration is measured before commencing a stripping operation).

### Organisms (i.e., Microbes)

Fermentation processes of the invention employ one or more microbes to produce one or more VOCs. In the present application, the terms “microbe” and “organism” are used interchangeably. Types of suitable organisms for production of a VOC by fermentation include bacteria, cyanobacteria, yeasts, and filamentous fungi. Examples of preferred organisms are an Acetobacter, Alcaligenes, Arthrobacter, Bacillus, Brevibacterium, Candida, Clostridium, Corynebacterium, Enterococcus. Erwinia, Escherichia, Flavobacterium, Gluconobacter, Hansenula, Klebsiella, Lactobacillus, Methylobacterium, Micrococcus, Mycobacterium, Nocardia, Paenibacillus Pichia, Pseudomonas, Rhodococcus, Saccharomyces, Salmonella, Thermoanaerobacter, Xanthobacter, and Zymomonas. A more preferred organism for producing 1-butanol by fermentation is a Clostridium, still more preferred is Clostridium acetobutylicum or Clostridium beijerinckii. A more preferred organism for producing ethanol by fermentation is Klebsiella, Saccharomyces, or Zymomonas.

An organism may be selected for use in a process of the present invention based on various criteria including 1) availability of biosynthetic machinery in the organism for producing a desired VOC product by fermentation; 2) ability of a particular organism to rapidly utilize a desirable nutrient carbon source such as, for example, carbohydrates, glycerol, or plant derived oils; and 3) growth tolerance of the organism to one or more VOCs produced by the organism. Certain species of Clostridium in a fermentation broth are able to produce a VOC mixture of acetone, butanol, and ethanol using starch as a feedstock, and are tolerant to levels of total VOCs up to approximately 2.0 wt %, i.e., a total of 2.0 grams of VOCs per 100 mL of the fermentation broth (weight/volume).

In describing the invention, for convenience, the shorter term “concentration” is sometimes used herein when referring to “time-averaged concentration” of one or more VOCs in a fermentation broth.

The term “nutrients feed” means a dry nutrient or a stream, solution, or suspension, comprising the dry nutrient.

The phrase “fluid communication” means engaging in, or being available for, receiving or sending a flow of a gas (e.g., vapor), liquid, or both. Fluid communication between any two elements (e.g., units or components) of an invention apparatus or process may be direct (e.g., via a direct connection between the two elements or via a fluid conduit (e.g., a pipe, a hose, and a duct) that provides direct connection between the two elements) or indirect (e.g., via one or more intermediary elements that are sequentially interposed between the two communicating elements). Selective fluid communication means fluid communication or being ready for fluid communication (e.g., by opening a valve). If two of units, components, or elements are in fluid communication with a common third unit, component, or element, then the two units, components, or elements are in fluid communication with each other. Any units, components, and elements described herein as being in fluid communication (direct or indirect) are also in operative connection unless stated otherwise. Fluid communication is by way of a generally leak free connection (less than 5 wt % leakage), preferably by way of a substantially leak free connection (less than 1 wt % leakage), more preferably by way of a leak free connection (less than 0.001 wt % leakage).

The term “operative connection” means direct or indirect (i.e., via the one or more intermediary elements as mentioned previously) and functional (i.e., operable for an intended purpose) attachment. Selective operative connection means operative connection or being ready for operative connection. An aperture of a vessel being operatively connected to an aperture of another vessel (i.e., the vessels being operatively connected to each other at their apertures) means being operatively connected to at least surfaces of the aperture; operatively connected to at least vessel exterior surfaces proximate to, and around, the aperture; operatively connected to at least vessel interior surfaces proximate to, and around, the aperture; or any combination thereof. Many ways of operatively connecting to apertures, inlets and outlets or otherwise are known and contemplated herein.

The term “operatively contacted” means direct or indirect and functional contact such as, for example, a shaft crossing through a lubricated shaft bearing and being operable to substantially freely rotate, or move forward and backward (e.g., in up and down directions in a vertically oriented shaft bearing) in the shaft bearing, the shaft bearing guiding the shaft to maintain the shaft in a desirable orientation (e.g., vertical).

Relative Arrangement of the Fermentor unit 200 and the VSS Unit (e.g., 10 of FIG. 1 or 100 of FIG. 2)

In the invention apparatus of the first embodiment, a fermentor unit 200 and a VSS unit (e.g., 10 of FIG. 1 or 100 of FIG. 2) may be, with respect to each other, in a vertical, horizontal, or in-between (e.g., diagonal) spatial arrangement. The fermentor unit 200 and VSS unit (e.g., 10 of FIG. 1 or 100 of FIG. 2) may be spaced apart from each other or contacting each other. In a vertical arrangement, the fermentor vessel (e.g., a standard fermentor vessel 211 (not shown) or a reboiled fermentor vessel 222 (not shown)) of the fermentor unit 200 is deployed approximately above, or at least is elevated with respect to, the stripper vessel (e.g., 78 of FIG. 1 and 178 of FIG. 2) of the VSS unit (e.g., 10 of FIG. 1 or 100 of FIG. 2). Thus, the first fluid aperture 271 (not shown) of the fermentor vessel (e.g., 211 and 222) may be located at a bottom portion 234 of the fermentor vessel (e.g., 211 and 222) and a third fluid aperture (e.g., 132) of the stripper vessel (e.g., 78 of FIG. 1 or 178 of FIG. 2) located at a top portion 54 or 175 of a stripper vessel (e.g., 78 of FIG. 1 and 178 of FIG. 2) of a VSS unit (e.g., 10 of FIG. 1 or 100 of FIG. 2).

Preferred, however, the fermentor unit 200 and the VSS unit (e.g., 10 of FIG. 1 or 100 of FIG. 2) are in a generally horizontal arrangement with respect to each other. In a horizontal arrangement, the fermentor vessel (e.g., 211 and 222) of the fermentor unit 200 may be deployed spaced apart from or approximately adjacent to the stripper vessel (e.g., 78 of FIG. 1 or 178 of FIG. 2) of the VSS unit (e.g., 10 of FIG. 1 or 100 of FIG. 2). Thus, the first fluid aperture 271 (not shown) and third fluid aperture 33 of FIG. 1 (not shown) may be located at side portions 230 (not shown) of the fermentor vessel (e.g., 211 and 222) and side portions 30 and 130 (not shown) of the stripper vessel (e.g., 78 of FIG. 1 or 178 of FIG. 2), respectively.

In any arrangement of a fermentor vessel (e.g., a standard fermentor vessel 211 (not shown) or a reboiled fermentor vessel 222 (not shown)) and stripper vessel (e.g., 78 of FIG. 1 and 178 of FIG. 2) in an invention apparatus, the first fluid aperture (e.g., 271) and third fluid aperture (e.g., 33 and 132) are in fluid communication with each other as described herein. When desired, one or more valves 17 may be deployed in fermentor vessel (e.g., 211 and 222), stripper vessel (e.g., 78 of FIG. 1 and 178 of FIG. 2), or between the fermentor vessel (e.g., 211 and 222) and stripper vessel (e.g., 78 of FIG. 1 and 178 of FIG. 2), or any combination thereof, to stop, start, or control the flow of a fermentation broth from or to the fermentor vessel (e.g., 211 and 222), the stripper vessel (e.g., 78 of FIG. 1 and 178 of FIG. 2), membrane vessel 811, if any, or from or to any other component of an invention apparatus.

Generally, gas apertures and vapor apertures in a vessel preferably occur in surfaces of an upper portion of the vessel. Fluid apertures for receiving a liquid into a vessel preferably occur in surfaces of a side portion or upper portion of the vessel. Fluid apertures for sending a liquid from a vessel preferably occur in surfaces of a side portion or lower portion of the vessel.

### Fermentor Unit 200

A fermentor unit 200 of the invention apparatus of the first embodiment comprises a fermentor vessel (e.g., a standard fermentor vessel 211 (not shown) or a reboiled fermentor vessel 222 (not shown)), as described herein. A standard fermentor vessel means a fermentor vessel that is operated at an atmospheric pressure, whereas a reboiled fermentor vessel means a fermentor vessel that is operated at a sub-atmospheric pressure. The fermentor unit 200 may further comprise other components such as, for example, a means for agitating a liquid 950, ultrafiltration membrane 822, fittings 218, gauges 219, valves 217, sensors 216, heat exchangers 214, and heating elements 213 and cooling elements 212 (all not shown). As described herein, the fermentor vessel (e.g., 211 and 222, not shown) defines at least two apertures (e.g., 271 and 272).

A fermentation broth may or may not be heated and/or agitated in a fermentor vessel (e.g., a standard fermentor vessel 211 (not shown) or a reboiled fermentor vessel 222 (not shown)). Preferably, the fermentation broth is heated or agitated, more preferably heated and agitated, in the fermentor vessel (e.g., 211 and 222). The fermentation broth may be heated by, for example, immersing a heating element (e.g., 213) or heat exchanger (e.g., 214), or both, therein. The fermentation broth or clarified liquid may be agitated by, for example, a stir motor (e.g., 225), shaft (e.g., 229), and impeller assembly (e.g., 228) or by bubbling an inert gas into a bottom portion (e.g., 234) of the fermentor vessel (e.g., 211 and 222) enclosed volumetric space.

In a fermentor unit 200, a standard fermentor vessel 211 or a reboiled fermentor vessel 222, also known as a steam-stripped fermentor vessel, is preferred. Fermentor vessels useful in the present invention are available from numerous commercial suppliers such as, for example, New Brunswick Scientific Company, Inc., Edison, N.J., USA.

A fermentation of an invention process is carried out using well-known methods. For example, first autoclave or in-situ sterilize the fermentor vessel (e.g., a standard fermentor vessel 211 (not shown) or a reboiled fermentor vessel 222 (not shown)) and then fill the resulting sterilized fermentor vessel (e.g., 211 and 222) with a nutrients feed, e.g., a batch of nutrient medium containing glucose or another assimilable carbohydrate such as starch or corn steep liquor. Preferably the nutrients feed is added to the fermentor vessel (e.g., 211 and 222) in a fed-batch or continuous mode. Inoculate the batch with an inoculum of mobile cells of a desired organism. Other additives, such as antifoaming agents, may be added to the broth to control foaming. An ultrafiltration membrane 822 (not shown) optionally may be disposed within the fermentor vessel (e.g., 211 and 222) to increase concentration of the cells in the fermentation broth.

Preferably, a flow of nitrogen sweep gas typically is swept through headspace of a reboiled fermentor vessel 222 (not shown) until the culture commences production of its own gases (CO₂ and H₂). At this point, the resulting batch process may be converted into a fed-batch or continuous process by periodically or continuously, respectively, adding a flow of nutrients feed containing carbohydrates and nutrients to the fermentation broth in the reboiled fermentor vessel 222. Later, a flow of bleed solution (i.e., a liquid portion of the fermentation broth) from the reboiled fermentor vessel 222 is started to maintain a constant liquid level in the reboiled fermentor vessel 222 and purge unwanted impurities such as non-volatile organic acids and other byproducts of the organism's metabolism that otherwise would accumulate in the reboiled fermentor vessel 222 over time. The cells remain in the reboiled fermentor vessel 222 while the liquid portion of the fermentation broth turns over.

### Vacuum Side Stripper (VSS) Unit

In the invention apparatus and process, a VSS unit (e.g., 10 of FIG. 1 or 100 of FIG. 2) is used to remove one or more VOCs from fermentation broth. A VSS unit (e.g., 10 of FIG. 1 or 100 of FIG. 2) is a horizontal multi-compartment distillation unit for stripping a side-stream at sub-atmospheric pressure and, preferably, elevated (i.e., above room temperature) temperature and under non-boiling conditions by injecting an inert gas (e.g., nitrogen) from a source of stripping gas (e.g., 11 and 111 (not shown)) through a gas aperture (e.g., 45 and 145, not indicated) of a stripper vessel (e.g., 78 of FIG. 1 and 178 of FIG. 2).

In general, the VSS unit (e.g., 10 of FIG. 1 or 100 of FIG. 2) comprises a stripper vessel (e.g., 78 of FIG. 1 and 178 of FIG. 2) that is a horizontal column having internal stripper compartments (e.g., 81-83 and 181-185) separated by partitions (e.g., 76 and 176) or baffles (not shown) designed to control countercurrent flow of liquid and vapor or gas through the VSS unit (e.g., 10 of FIG. 1 or 100 of FIG. 2). In an aspect of the first embodiment, the invention apparatus further comprises a second VSS unit (e.g., another 10 of FIG. 1 or 100 of FIG. 2), the second VSS unit (e.g., another 10 of FIG. 1 or 100 of FIG. 2) independently having an enclosed volumetric space of a second stripper vessel that is in sequential operative connection to, and fluid communication with, the enclosed volumetric space of the stripper vessel of the stripper vessel (e.g., 78 of FIG. 1 and 178 of FIG. 2) of the (primary) VSS unit (e.g., 10 of FIG. 1 or 100 of FIG. 2), and enclosed volumetric space of a fermentor vessel (e.g., a standard fermentor vessel 211 (not shown) or a reboiled fermentor vessel 222 (not shown)) of the fermentor unit 200 (not shown) of the first embodiment. An invention apparatus having three or more such independent VSS units is also contemplated wherein the three or more VSS units may be in parallel or serial operative connection to, and fluid communication with, the fermentor unit 200.

A VSS unit (e.g., 10 of FIG. 1 or 100 of FIG. 2) may further comprise other components such as, for example, fittings 218, valves 217, gauges 219, sensors 216, heat exchangers 214, heating elements 213 and cooling elements 212 (all not shown) as described above for a fermentor unit 200 (not shown). As described herein, the stripper vessel (e.g., 78 of FIG. 1 and 178 of FIG. 2) defines apertures (e.g., 132 and 141), which may be disposed in the stripper vessel (e.g., 78 of FIG. 1 and 178 of FIG. 2) so as to be proximal to, or distal from, each other, or any combination thereof. Apertures (e.g., 132 and 141) in the stripper vessel (e.g., 78 of FIG. 1 and 178 of FIG. 2) provide access to the interior (e.g., 57 and 156) of the stripper vessel (e.g., 78 of FIG. 1 and 178 of FIG. 2) for adding and removing contents of the stripper vessel (e.g., 78 of FIG. 1 and 178 of FIG. 2) and attaching other components of the invention apparatus and support structures (not shown) to the stripper vessel (e.g., 78 of FIG. 1 and 178 of FIG. 2). Examples of stripper vessel contents are the same as described above for fermentor vessel contents.

VSS units (e.g., 10 of FIG. 1 or 100 of FIG. 2), of the agitation type and circulation type, are valuable means for stripping one or more VOCs from fermentation broth. Accordingly, another embodiment of the present invention is an apparatus, the apparatus comprising a VSS unit (e.g., 10 of FIG. 1 or 100 of FIG. 2) as described herein.

Where a stripper vessel (e.g., 78 of FIG. 1 and 178 of FIG. 2) has disposed therein three or more stripper compartments (e.g., 81 to 83 and 181 to 185), that is when there is one or more intermediate stripper compartments (e.g., 82 and 182-184), the three or more stripper compartments may be in a linear or non-linear (e.g., perpendicular) arrangement.

Vertical partition members (e.g., 76 and 176) may comprise a unified part of the stripper vessel (e.g., a stripper vessel that has been formed with inwardly protruding horizontal portions of the stripper vessel itself). Vertical partition members (e.g., 76 and 176) may be suspended inside the stripper vessel, but without touching the stripper vessel (e.g., on a generally horizontal rod 97 (not shown) that traverses through a center aperture 98 in each of the vertical partition members (e.g., 76 and 176) and generally spans a length of the stripper vessel between proximal 90 and distal ends 89 (not shown) thereof).

Preferably, a VSS unit (e.g., 10 of FIG. 1 or 100 of FIG. 2) comprises at least one means 900 for contacting a liquid to a vapor, the means 900 for contacting a liquid to a vapor being operatively contacted to a stripper vessel (e.g., 78 of FIG. 1 and 178 of FIG. 2) of the VSS unit (e.g., 10 of FIG. 1 or 100 of FIG. 2) at a first agitation aperture (not indicated) of the stripper vessel (e.g., 78 of FIG. 1 and 178 of FIG. 2) and being in fluid communication with a stripper compartment interior. More preferably, the VSS unit (e.g., 10 of FIG. 1 or 100 of FIG. 2) comprises means 900 for contacting a liquid to a vapor for each stripper compartment (e.g., 81-83 and 181-185) and the stripper vessel (e.g., 78 of FIG. 1 and 178 of FIG. 2) further defining agitation apertures 35 to 37 and 150 to 154/135-139 (not indicated) such that there is at least one agitation aperture for each stripper compartment (e.g., 81-83 and 181-185), each means 900 for contacting a liquid to a vapor being operatively contacted to the stripper vessel (e.g., 78 of FIG. 1 and 178 of FIG. 2) at a different agitation aperture 35 to 37 and 150 to 154/135-139 and being in fluid communication with a different stripper compartment interior (not indicated).

When a liquid (e.g., fermentation broth or a clarified liquid derived therefrom) is added to one stripper compartment (e.g., 81-83 and 181-185) of the stripper vessel (e.g., 78 of FIG. 1 and 178 of FIG. 2) of the VSS unit (e.g., 10 of FIG. 1 or 100 of FIG. 2), the liquid will typically flow in fluid communication through liquid trafficking conduits (e.g., 94 and 194), liquid trafficking apertures 4, or any combination thereof to the other stripper compartments of the stripper vessel (e.g., 78 of FIG. 1 and 178 of FIG. 2). The liquid will thus flow around, through, or around and through a vertical partition member (e.g., 76 and 176) dividing the stripper compartments.

In any particular stripper compartment (e.g., 81-83 and 181-185) having a liquid disposed therein, there preferably will be a liquid space (i.e., the space where the liquid is) and a headspace (containing gas, vapor, or both, and during agitation or circulation of the liquid, liquid droplets or splashes) above the liquid space. The liquid spaces will be in sequential liquid communication with each other and the headspaces will be in sequential gas/vapor communication with each other.

Stripper compartments (e.g., 81-83 and 181-185) in a stripper vessel (e.g., 78 of FIG. 1 and 178 of FIG. 2) may be of same or different volumes, or a combination thereof. Preferably, the stripper compartments (e.g., 81-83 and 181-185) of a stripper vessel (e.g., 78 of FIG. 1 and 178 of FIG. 2) are of generally same volumes.

Depending on a particular fermentation process (e.g., particular organism and its sensitivity to product inhibition effects), a stripping operation may be started at any time-averaged total concentration of VOC(s) in a fermentation broth in a fermentor vessel (e.g., a standard fermentor vessel 211 (not shown) or a reboiled fermentor vessel 222 (not shown)) or in a stripper vessel (e.g., 78 of FIG. 1 and 178 of FIG. 2).

For illustration, a stripping operation of with VSS unit 10 comprises an agitation (liquid) operation, vacuum operation, gas stripping operation, heating operation, or any combination thereof. Preferably, a stripping operation of the VSS unit 10 comprises agitation, vacuum, gas stripping, and heating operations.

During the agitation operation of the VSS unit 10, preferably fermentation broth, or a clarified liquid derived therefrom, is stirred in, or circulated to nozzles in headspaces of, at least one stripper compartment (e.g., 81-83 and 181-185) of a stripper vessel (e.g., 78 of FIG. 1 and 178 of FIG. 2); more preferably each stripper compartment (e.g., 81-83 and 181-185).

During the vacuum operation of the VSS unit 10, a vacuum from a vacuum source 5 may sequentially draw a stream of fermentation broth, or a clarified liquid derived therefrom, from the fermentor vessel (e.g., 211 and 222), through the first fluid aperture (e.g., 271), third fluid aperture (e.g., 132) and into at least one stripper compartment (e.g., 81-83 and 181-185) of the second interior of the stripper vessel (e.g., 78 of FIG. 1 and 178 of FIG. 2).

During the stripping gas injecting operation of the VSS unit 10 or 100, the gas aperture (e.g., 45 and 145) of the stripper vessel (e.g., 78 of FIG. 1 and 178 of FIG. 2) is operatively connected to a stripping gas source (e.g., 11 and 111, not shown); e.g., source of fermentation off-gases such as CO₂ and H₂ and/or source of nitrogen or argon inert gases). The gas aperture (e.g., 45 and 145) and vapor aperture (e.g., 46, not indicated and 141) may be proximate to or distal from each other in the stripper vessel (e.g., 78 of FIG. 1 and 178 of FIG. 2). Preferably, the gas aperture (e.g., 45 and 145, both not indicated) is in direct fluid communication with a distal stripper compartment (e.g., 83 and 185) and the vapor aperture (e.g., 46 and 141) is in direct fluid communication with a proximal stripper compartment (e.g., 81 and 181) of the interior (e.g., 57 and 156) of the stripper vessel (e.g., 78 of FIG. 1 and 178 of FIG. 2) so as to produce a sequential flow of injected stripping gas from stripping gas source (e.g., 11 and 111) through each stripper compartment (e.g., 81-83 and 181-185) and out the vapor aperture (e.g., 46 and 141). Alternatively, such sequential flow may be produced from a gas aperture (e.g., 45 and 145) that is proximate to the vapor aperture (e.g., 46 and 141) by injecting a stripping gas from a source of stripping gas (e.g., 11 and 111) from the gas aperture (e.g., 45 and 145) through the stripper vessel interior (e.g., 57 and 156) and into the distal stripper compartment (e.g., 83 and 185), wherein the injected stripping gas is released and sequentially flows to the vapor aperture (e.g., 46 and 141).

During the heating operation of the VSS unit 10, a heating element 213 or heat exchanger 214 immersed in, or in operative connection beneath, the stripper vessel (e.g., 78 of FIG. 1 and 178 of FIG. 2) is actuated.

During the stripping operation of the VSS unit 10, the fermentation broth or clarified liquid is thereby stripped in the stripper vessel (e.g., 78 of FIG. 1 and 178 of FIG. 2) to produce a wet VOC vapor (not shown) comprising water vapor (as mentioned previously, the fermentation broth or clarified liquid is aqueous) and vapor of at least one VOC. Such wet VOC vapor flows from the stripper vessel (e.g., 78 of FIG. 1 and 178 of FIG. 2) through the vapor aperture (e.g., 46, not indicated, and 141).

Preferably, the stripping operation of a VSS unit 10 comprises an agitation, vacuum, stripping gas injecting, or heating operation, or any combination thereof. More preferably, stripping operation of the VSS unit 10 comprises an agitation, vacuum, stripping gas injection, and heating operation.

Compared to a conventional packed column or trayed column stripper (not shown), a VSS unit (e.g., 10 of FIG. 1 or 100 of FIG. 2) is less prone to become fouled with fermentation broth solids (e.g., a biomass of cells of an organism). Stripping temperature in a VSS unit (e.g., 10 of FIG. 1 or 100 of FIG. 2) does not need to be kept below the maximum temperature an organism can tolerate provided that temperature of stripped liquid returned to a fermentor vessel (e.g., a standard fermentor vessel 211 (not shown) or a reboiled fermentor vessel 222 (not shown)) preferably is not hot enough to substantially kill (i.e., a kill rate of greater than 10% per day) cells of the organism in the fermentation broth in the fermentor vessel (e.g., 211 and 222). Stripping temperature may be controlled as needed using heat exchangers 214, as described herein, to improve energy efficiency if the invention process.

Preferably, counter-flowing gas and liquid phases are contacted or mixed in each stripper compartment (e.g., 81-83 and 181-185) of the stripper vessel (e.g., 78 of FIG. 1 and 178 of FIG. 2) to promote the stripping process. For example, the injected inert gas entrains VOCs by flowing in a direction from a distal stripper compartment (e.g., 83 and 185) of the stripper vessel (e.g., 78 of FIG. 1 and 178 of FIG. 2) sequentially through any intermediate stripper compartments (e.g., 82 and 182-184) to a proximal stripper compartment (e.g., 81 and 181) and out a vapor aperture (e.g., 46, not shown, and 141) in the stripper vessel (e.g., 78 of FIG. 1 and 178 of FIG. 2), the vapor aperture (e.g., 46 and 141) being in fluid communication with the proximal stripper compartment (e.g., 81 and 181). At the same time, a feed of fermentation broth, or a clarified liquid derived therefrom, is fed into the proximal stripper compartment (81 and 181) and flows, in a direction counter to the direction of inert gas flow, sequentially through any intermediate stripper compartments (e.g., 82 and 182-184) to the distal stripper compartment (e.g., 83 and 185) and out another fluid aperture (e.g., 34 and 134, both not shown) in the stripper vessel (e.g., 78 of FIG. 1 and 178 of FIG. 2), the other fluid aperture (e.g., 34 and 134) being in fluid communication with the distal stripper compartment (e.g., 83 and 185) and, preferably, fermentation broth in a fermentor vessel (e.g., a standard fermentor vessel 211 (not shown) or a reboiled fermentor vessel 222 (not shown)) of a fermentor unit 200.

Counter-flowing gas and liquid phases can be contacted or mixed in each stripper compartment (e.g., 81-83 and 181-185) by a means 900 for contacting a liquid to a vapor, as described later. Normally, satisfactory stripping performance is obtained with 3 to 10 stripper compartments (e.g., 81-83 and 181-185) in the VSS unit (e.g., 10 of FIG. 1 or 100 of FIG. 2). (The VSS unit (e.g., 10 of FIG. 1 or 100 of FIG. 2) differs from the type of horizontal distillation column described by Markels and Drew [“A Horizontal Fractionating Device,” Ind. Eng. Chem., vol. 51, pp. 619-624 (1959)] because, unlike the Markel and Drew design, the VSS unit (e.g., 10 of FIG. 1 or 100 of FIG. 2) does not contain packing materials within at least liquid spaces (e.g., indicated by bottom-half, gray background portion of interior (not indicated) of stripper vessel 178 in FIG. 2) of the stripper compartments (e.g., 81-83 and 181-185), preferably does not contain a substantial amount of packing materials within liquid spaces and headspaces (e.g., indicated by top-half, white background portion of interior (not indicated) of stripper vessel 178 in FIG. 2) of the stripper compartments (i.e., preferably each stripper compartment does not contain a substantial amount, meaning taking up more than 5% of stripper compartment volume, of packing materials), more preferably does not contain any amount of packing materials within liquid spaces and headspaces of the stripper compartment (i.e., preferably each stripper compartment does not contain any amount, meaning about 0% of stripper compartment volume, of packing materials). The VSS unit (e.g., 10 in FIG. 1 and 100 in FIG. 2) virtually eliminates the potential for fouling of the internals due to plugging of the packing material with fermentation broth solids.

The amount of stripping gas entering a stripper vessel (e.g., 78 of FIG. 1 and 178 of FIG. 2) of a VSS unit (e.g., 10 of FIG. 1 or 100 of FIG. 2) may be adjusted to allow operation of the VSS unit (e.g., 10 of FIG. 1 or 100 of FIG. 2) at a pressure that is above a minimum pressure a microbe can tolerate, and at a temperature below the maximum temperature the microbe can tolerate. These are only estimates of a minimum pressure limit The actual minimum pressure limit will depend upon the type of organism. For example, Gram-negative bacterial organisms will be less tolerant of a sudden reduction in pressure compared to Gram-positive bacterial organisms. This is because Gram-positive microbes have a thicker cell wall. The Clostridium organism is a Gram-positive, spore forming organism and is more robust than most Gram-negative organisms, such as E. coli, in an ABE fermentation.

Preferably, a stripper vessel (e.g., 78 of FIG. 1 and 178 of FIG. 2) and a fermentor vessel (e.g., a standard fermentor vessel 211 (not shown) or a reboiled fermentor vessel 222 (not shown)) each also define return fluid apertures (e.g., 34 and 134, both not shown), the stripper vessel (e.g., 78 of FIG. 1 and 178 of FIG. 2), at the stripper vessel return fluid aperture (e.g., 34 and 134), being in further operative connection to, and fluid communication with, the fermentor vessel (e.g. 211 and 222) at the fermentor vessel return fluid aperture (e.g., 264, not shown) such as by a return line (e.g., 32 and 263, not shown).

A skilled artisan would know that one or more additional units and components may be deployed in an invention apparatus of the first embodiment and process of the second embodiment to accomplish certain further processing operations. Examples of such additional units and components are described below.

### Additional Components

In some embodiments, an apparatus of the present invention further comprises, among other things, one or more additional components selected from the group consisting of: a blower; a first vacuum pump/compressor; a second vacuum pump/compressor; a vapor/liquid condenser; a decanter; a liquid-liquid extractor; a means for separating at least one volatile organic compound from water (means for separating); a dividing-wall distillation column (DWC) unit; an ultrafiltration membrane; and an membrane vessel, wherein the blower, first vacuum pump/compressor, second vacuum pump/compressor, vapor/liquid condenser, decanter, liquid-liquid extractor, means for separating, and DWC unit independently are in operative connection to, and fluid communication with the vapor aperture of the stripper vessel; the ultrafiltration membrane being disposed within the membrane vessel or the membrane vessel is absent and the ultrafiltration membrane being disposed within the fermentor vessel; the membrane vessel being in operative connection to, and fluid communication with, the first fluid aperture of fermentor vessel and the third fluid aperture of the stripper vessel; fluid communication being established between the fermentor vessel, stripper vessel and the one or more additional components. In a preferred embodiment, the means for separating at least one volatile organic compound from water comprises a pressure-swing adsorption (PSA) unit.

In still another preferred embodiment, the one or more additional components comprise the PSA unit and the DWC unit, the vapor aperture of the stripper vessel being in sequential operative connection to, and fluid communication with, the PSA unit and DWC unit.

In still another preferred embodiment, the apparatus further comprises the ultrafiltration membrane.

In still another preferred embodiment, the apparatus further comprises the first vacuum pump/compressor, second vacuum pump/compressor, vapor/liquid condenser, decanter, PSA unit, and DWC unit, the vapor aperture of the stripper vessel being in sequential operative connection to, and fluid communication with, the first vacuum pump/compressor, second vacuum pump/compressor, vapor/liquid condenser, decanter, PSA unit, and DWC unit.

In still another preferred embodiment, the stripper vessel and fermentor vessel each further define a return fluid aperture, the return fluid aperture of the stripper vessel being in operative connection to, and fluid communication with, the return fluid aperture of the fermentor vessel.

Examples of a means for separating at least one volatile organic compound from water are a PSA unit 400 (not shown); a conventional adsorbent unit 401 (not shown), wherein at least one bed of water adsorbent (e.g., silica gel or a zeolite such as a 3 angstrom molecular sieve) is used for adsorbing water from a liquid feed of wet VOC(s) and producing a liquid stream of substantially dry VOC(s); and a conventional azeotropic distillation unit 402 (not shown) employing an organic azeotropic drying agent (e.g., toluene or ethyl acetate), which is also generally known as an entrainer.

### PSA Unit 400

Preferably, the means for separating VOC(s) from water is one or more PSA units 400 (not shown). PSA units 400 and PSA cycles are known and described by in Gas Separation by Adsorption Processes, by Ralph T. Yang, World Scientific Publishing Company, Pte. Ltd., Singapore (USA office River Edge, New Jersey), 1997. For purposes of the instant invention, a PSA unit 400 comprises a vaporizer subunit 444 (not shown) and a separation subunit 445 (not shown), each subunit having a fluid inlet and fluid outlet.

### DWC Unit 500

A DWC unit 500 (not shown) is described in Perry's Chemical Engineers' Handbook by Don W. Green and Robert H. Perry, 8^{th} edition, 2007, McGraw-Hill Professional, New York, N.Y., USA. A DWC unit is useful for separating two or more VOCs in a mixture thereof from each other, preferably wherein mixture the two or more VOCs is substantially dry. When four or more VOCs are produced in an invention process, a series of two or more conventional distillation columns, DWC units 500, or any combination thereof, may be readily arranged to separate the four or more VOCs from each other.

### Dual-Function Column 300

The invention apparatus and process may further comprise a dual-function column 300 (not shown). A dual-function column 300 in an invention apparatus and process serves at least two purposes: 1) to concentrate VOC vapors leaving a fermentor vessel (e.g., a reboiled fermentor vessel 222 (not shown)) or stripper vessel (e.g., 78 of FIG. 1 and 178 of FIG. 2); and 2) to strip VOCs from an aqueous layer fed to the dual-function column from a decanter 650 before the aqueous layer is, for example, returned to the fermentor vessel (e.g., 222, not shown) or sent to waste. The dual-function column 300 may be packed with conventional distillation packing, with structured distillation packing, or it may have distillation trays disposed therein. A dual-function column may have a top portion that may function as a mist eliminator.

### Ultrafiltration Membrane 822 and Ultrafiltration Membrane Unit 800

An ultrafiltration membrane 822 (not shown) is an assembly of conventional membrane modules such as, for example, hollow-fiber membrane modules or flat-sheet membrane modules that are autoclavable or in-situ sterilizable. An ultrafiltration membrane 822 is preferably sterilized prior to starting a fermentation process and monitored for potential fouling during the fermentation process. Examples of such membrane modules useful in the present invention are the hollow-fiber membrane modules manufactured by Zenon, a subsidiary of General Electric. General aspects of the application of ultrafiltration membranes for water filtration in bioreactor operations are described by Yang, Cicek, and Ilg [“State-of-the-Art of Membrane Bioreactors: Worldwide Research and Commercial Applications in North America,” J. Membr. Sci., vol. 270, pp. 201-211 (2006)] and are contemplated for the present invention apparatus and process.

An invention apparatus and process optionally may further comprise, among other things, an ultrafiltration membrane 822 (not shown), which may be deployed within a fermentor vessel (e.g., a standard fermentor vessel 211 (not shown) or a reboiled fermentor vessel 222 (not shown)) or in a membrane vessel 811 (not shown) that comprises an ultrafiltration membrane unit 800 (not shown). Such membrane vessel 811 defines at least two apertures, feed fluid aperture 837 and bleed fluid aperture 838, and preferably a third aperture, a vacuum aperture 839 (all not shown). The membrane vessel 811 is interposed between, and in operative connection to, and fluid communication with, the fermentor vessel (e.g., 211 and 222) and a stripper vessel (e.g., 78 of FIG. 1 and 178 of FIG. 2) of a VSS unit (e.g., 10 of FIG. 1 or 100 of FIG. 2) as described elsewhere.

### Mechanical Vapor Recompression

Operation of the two sequentially connected vacuum pump/compressors 370 and 380 (both not shown) is commonly called mechanical vapor recompression, and it serves to improve energy efficiency of a (boiling) fermentation process. Alternatively, overheads vapors from a stripper vessel (e.g., 78 of FIG. 1 and 178 of FIG. 2) of a VSS unit (e.g., 10 of FIG. 1 or 100 of FIG. 2), a dual-function column 300 (not shown), or a reboiled fermentor vessel 222 (not shown) may be compressed up to atmospheric pressure in a single vacuum pump/compressor 370.

### Means 900 for Contacting a Liquid to Vapor

A means 900 for contacting a liquid to a vapor (not shown) is a means 950 for agitating a liquid (not shown) or a means 960 for circulating a liquid (not shown). The means 950 for agitating a liquid means an apparatus for stiffing, bumping, or otherwise moving a liquid in a fermentor vessel (e.g., a standard fermentor vessel 211 (not shown) or a reboiled fermentor vessel 222 (not shown)) of a fermentor unit 200 (not shown) or a stripper compartment (e.g., 81 to 83 and 181 to 185) of an enclosed volumetric space of the stripper vessel (e.g., 78 of FIG. 1 and 178 of FIG. 2) of a VSS unit (e.g., 10 of FIG. 1 or 100 of FIG. 2). A preferred means 950 for agitating a liquid is a conventional stir motor (e.g., 25), stir shaft (e.g., 29), and impeller (e.g., 28) of a stir assembly 27. A preferred means 960 for circulating a liquid is fluid conduit (e.g., 164, 166, and 163), liquid pump (e.g., 165), and nozzle (e.g., 162) assembly depicted in FIG. 2. Examples of suitable liquid pumps (e.g., 165) are disc-type pumps manufactured by the Discflo Corporation Inc., Santee, Calif., USA and reduced-shear recessed-impeller centrifugal pumps such as those manufactured by Durco International Inc., Flowserve Corporation, Irving, Tex., USA).

### General Fermentation Processes

The invention apparatus and process decouples fermentation process conditions from stripper process conditions, thus allowing operation of both fermentation and stripping at their respective most effective conditions. More specifically, the invention apparatus and process allows operation of a fermentor vessel (e.g. standard fermentor vessel 211, not shown) at atmospheric pressure (thereby avoiding difficulties operating a large fermentor vessel at sub-atmospheric pressures including requirements for expensive vessel construction and the potential for air infiltration and contamination of the fermentation broth by a competing microbe present in the outside environment), and at the same time allows operation of VSS unit (e.g., 10 of FIG. 1 or 100 of FIG. 2) at a different pressure and temperature optimized for good stripping performance without incurring damage to the microbes of the fermentation broth.

Injection of a stripping gas into a stripper vessel (e.g., 78 of FIG. 1 and 178 of FIG. 2) may be adjusted to controllably maintain pressure in the stripper vessel (e.g., 78 of FIG. 1 and 178 of FIG. 2) above a minimum pressure that could cause cell damage due to sudden pressure change experienced by microbes entering the stripper vessel (e.g., 78 of FIG. 1 and 178 of FIG. 2) in a stream of fermentation broth from a fermentor vessel (e.g., a standard fermentor vessel 211 (not shown) or a reboiled fermentor vessel 222 (not shown)). At the same time temperature may be controllably maintained at or below the maximum temperature (typically 35° C.) a particular microbe can tolerate. Thus, a VSS unit (e.g., 10 of FIG. 1 or 100 of FIG. 2) is operated at most-effective conditions above the minimum pressure and below the maximum temperature a microbe can tolerate. If no stripping gas is injected into a stripper vessel (e.g., 78 of FIG. 1 and 178 of FIG. 2) of the VSS unit (e.g., 10 of FIG. 1 or 100 of FIG. 2), the VSS unit (e.g., 10 of FIG. 1 or 100 of FIG. 2) preferably operates at boiling conditions for a fermentation broth at a given operating pressure in the stripper vessel (e.g., 78 of FIG. 1 and 178 of FIG. 2).

Compared to an atmospheric-pressure gas-stripping operation, sub-atmospheric pressure operation of the VSS unit (e.g., 10 of FIG. 1 or 100 of FIG. 2) allows use of less stripping gas and improved stripping effectiveness. For gas stripping, effectiveness of the relative volatility driving force is reduced by the ratio of water vapor pressure divided by total pressure. Thus, operating a VSS unit (e.g., 10 of FIG. 1 or 100 of FIG. 2) at sub-atmospheric pressure is more effective in terms of stripping performance than operating a VSS unit (e.g., 10 of FIG. 1 or 100 of FIG. 2) at atmospheric pressure.

The previously described horizontal multi-compartment design of a stripper vessel (e.g., 78 of FIG. 1 and 178 of FIG. 2) of the VSS unit (e.g., 10 of FIG. 1 or 100 of FIG. 2) provides for much lower vapor-traffic pressure drop between a fermentor vessel (e.g., a standard fermentor vessel 211 (not shown) or a reboiled fermentor vessel 222 (not shown)) and the stripper vessel (e.g., 78 of FIG. 1 and 178 of FIG. 2) compared to what would be expected between the fermentor vessel (e.g. 211 and 222) and a conventional packed tower 1001 (not shown) or trayed stripping tower 1002 (not shown) under similar process conditions.

The horizontal multi-compartment design also facilitates injection of stripping gas using a blower 350 (not shown), in operative connection to, and fluid communication with, a vapor aperture (e.g., 46, not shown, and 141) of the stripper vessel (e.g., 78 of FIG. 1 and 178 of FIG. 2). The blower 350 generates very low head pressure, as the stripping gas does not need to overcome significant pressure drop on its way through stripper vessel (e.g., 78 of FIG. 1 and 178 of FIG. 2). This allows use of an inexpensive blower 350 with minimal energy consumption.

The horizontal multi-compartment stripper vessel design also is more resistant to fouling compared to conventional packed tower 1001 and trayed tower 1002 side-stripper designs. A stripper vessel (e.g., 78 of FIG. 1 and 178 of FIG. 2) of a VSS unit (e.g., 10 of FIG. 1 or 100 of FIG. 2) can tolerate much greater accumulation of bio-mass deposits on the internal surfaces (e.g., 62 and 61) before mass-transfer performance is significantly negatively impacted.

When an ultrafiltration membrane 822 (not shown) is deployed in an invention apparatus and process, preferably the ultrafiltration membrane 822 is operated without a substantial increase (e.g., substantial increase meaning an increase of 20% or more) in the concentration of organism cells within a fermentation broth in a fermentor vessel (e.g., a standard fermentor vessel 211 (not shown) or a reboiled fermentor vessel 222 (not shown)) or membrane vessel 811 (not shown) in order to minimize fouling of the ultrafiltration membrane 822. Preferably, to avoid potential for damage to an organism due to sudden change in pressure, the organism cells are confined within the fermentor vessel (e.g., 211 and 222) or membrane vessel 811 and pressure within the fermentor vessel (e.g., 211 and 222) or membrane vessel 811 is maintained at or above a minimum pressure (e.g., at or above 0.8 atm for Clostridium beijerinckii) that is tolerated by the organism. More preferably, an ultrafiltration membrane 822 is operated at cell concentrations in fermentation broth in a fermentor vessel (e.g., a standard fermentor vessel 211 (not shown) or a reboiled fermentor vessel 222 (not shown)) having the ultrafiltration membrane 822 disposed therein or in a membrane vessel 811) within the range of 10 g/L to 80 g/L, preferably within the range of 20 g/L to 50 g/L.

Because a clarified liquid derived from fermentation broth contains no or only a small fraction of organism cells, temperature of the clarified liquid in the stripper vessel (e.g., 78 of FIG. 1 and 178 of FIG. 2) during stripping may be higher than a maximum temperature that the organism can tolerate (i.e., remain viable at). Stripped clarified liquid may be returned to a fermentor vessel (e.g., a standard fermentor vessel 211 (not shown) or a reboiled fermentor vessel 222 (not shown)) or sent to waste. Clarified liquid that is returned to the fermentor vessel (e.g., 211 and 222) preferably enters the fermentor vessel (e.g., 211 and 222), and contacts residual fermentation broth in the fermentor vessel (e.g., 211 and 222), at a temperature that is not too hot for the organism, i.e., a temperature that will not kill more than 10% of cells of the organism.

Some fermentation processes will produce an aqueous mixture comprising water and one or more water miscible VOCs (e.g., ethanol, 2-propanol, 1,3-propanediol, 1,4-butanediol, and acetone). A water miscible VOC is a VOC that is completely miscible in water in the absence of another, less water miscible VOC or an inorganic solute dissolved in water. Accordingly, a means 777 for inducing phase separation of a wet VOC liquid mixture into an organics liquid layer and an aqueous liquid layer (not shown) (means 777 for inducing phase separation), wherein the means 777 for inducing phase separation is at least in fluid communication with the aqueous mixture, which may be contained in, for example, a decanter 650. Examples of a means for inducing phase separation are dissolving an inorganic salt (not shown) (e.g., Nal) in the aqueous mixture to decrease solubility of the water miscible VOCs in water and a liquid-liquid extractor 700 (not shown), optionally also employing a liquid-liquid mixer 709 (not shown) in-line before a decanter 650 (not shown).

While various conventional stripping alternative methods (e.g., pervaporation using membranes and the use of liquid-liquid extraction) can be used for removing VOC(s) from fermentation broth, stripping with the VSS unit (e.g., 10 of FIG. 1 or 100 of FIG. 2) of the invention apparatus of the first embodiment is superior. For example, gas-injection could be practiced using a conventional packed tower 1001 or trayed tower 1002 side-stripper in place of a VSS unit (e.g., 10 of FIG. 1 or 100 of FIG. 2). However, the conventional packed tower 1001 or trayed tower 1002 side-stripper can be operated either at atmospheric pressure with a stripping gas (e.g., nitrogen gas) or under vacuum at sub-atmospheric pressure and boiling conditions, but without a stripping gas. The conventional packed tower 1001 or trayed tower 1002 side-stripper cannot be operated at sub-atmospheric pressure and non-boiling conditions with a stripping gas. In contrast, the VSS units (e.g., 10 and 100) depicted in FIGS. 1 and 2 can be operated at sub-atmospheric pressure and non-boiling conditions with a stripping gas and thereby achieves significant benefits that cannot be obtained with the conventional packed or trayed tower side-stripper.

The invention apparatus and process may be used with any fermentation process that produces one or more VOCs. An invention process of the second embodiment is further illustrated below in relation to an ABE fermentation process.

Illustration with acetone-(1-butanol)-ethanol (ABE) Fermentation

For production of 1-butanol in a typical ABE fermentation broth using Clostridium beijerinckii as the organism, and a standard fermentor vessel (e.g., 211, not shown) one or more of the following operation parameters are preferred:
a maximum concentration of total VOCs of about 1.2 wt %, i.e., a maximum of 1.2 grams of total VOCs (i.e., weight 1-butanol plus weight ethanol plus weight acetone plus weight of other VOC(s), if any) per 100 mL of the fermentation broth;
productivity of the organism in a commercially desirable range of from about 0.5 grams to about 4 grams of butanol produced per liter of fermentation broth per hour;
stripping is started when time-averaged total concentration of VOC(s) in a fermentation broth in the standard fermentor vessel (e.g., 211) is at least 0.1 grams total weight of the at least one VOC per 100 milliliters of fermentation broth (i.e., at least 0.1 weight percent (wt %)); more preferably from 0.1 wt % to 2.0 wt %; still more preferably about 0.3 wt % to 0.4 wt %;
when the total VOCs concentration in a broth in the standard fermentor vessel (e.g., 211, not shown) reaches a level of, or, preferably, about 1.0 wt %, a stream of ABE fermentation broth is pumped from the standard fermentor vessel (e.g. 211) to a stripper vessel (e.g., 78 of FIG. 1 and 178 of FIG. 2) of a VSS unit (e.g., 10 of FIG. 1 or 100 of FIG. 2), and, optionally, the ABE fermentation broth is pumped back to the standard fermentor vessel (e.g. 211);
a stripping-gas flow rate is adjusted to control the time-averaged concentration of total VOCs (i.e., 1-butanol plus VOC co-products) in the liquid phase of the ABE fermentation broth in the stripper vessel (e.g., 78 of FIG. 1 and 178 of FIG. 2), the standard fermentor vessel (e.g. 211), or both, more preferably time-averaged total concentration of VOC(s) in a fermentation broth in the standard fermentor vessel (e.g. 211) is maintained at less than 0.5 wt %, more preferably in a range of from 0.8 wt % to 1.2 wt % during stripping;
a level of the ABE fermentation broth in the standard fermentor vessel (e.g. 211) is adjusted upward or downward as needed by manipulating a rate of flow of nutrients feed into the standard fermentor vessel (e.g. 211), a bleed of ABE fermentation broth from the standard fermentor vessel (e.g. 211) to waste, or a stream of broth from the standard fermentor vessel (e.g. 211) to the stripper vessel (e.g., 78 of FIG. 1 and 178 of FIG. 2), or any combination thereof; similarly, a level of broth in the stripper vessel (e.g., 78 of FIG. 1 and 178 of FIG. 2) may be adjusted upward or downward as needed by manipulating a rate of flow of broth from the standard fermentor vessel (e.g. 211) into the stripper vessel (e.g., 78 of FIG. 1 and 178 of FIG. 2), a rate of flow of stripped broth leaving the stripper vessel (e.g., 78 of FIG. 1 and 178 of FIG. 2) and returning to the standard fermentor vessel (e.g. 211), a rate of flow of a bleed of stripped broth leaving the stripper vessel (e.g., 78 of FIG. 1 and 178 of FIG. 2) and sent to waste, or any combination thereof;
a preferred concentration of cells of an organism in an ABE fermentation broth in the standard fermentor vessel (e.g., 211) is from grams per liter (g/L) to 120 g/L, more preferably from 40 g/L to 80 g/L;
when there is an unfiltered ABE fermentation broth in a stripper vessel (e.g., 78 of FIG. 1 and 178 of FIG. 2), the unfiltered ABE fermentation broth in the stripper vessel (e.g., 78 of FIG. 1 and 178 of FIG. 2) is maintained at a temperature of from 20 degrees Celsius (° C.) to 50° C., more preferably at 35° C.;
the VSS unit (e.g., 10 of FIG. 1 or 100 of FIG. 2) is operated at pressures of from about 0.13 atm to about 0.46 atm (i.e., from about 100 mm Hg to 350 mm Hg);
when a mechanical vapor recompression means is in operative connection to vapor aperture 46 or 141 (not shown) of a stripper vessel (respectively, 78 of FIG. 1 and 178 of FIG. 2), a first vacuum pump/compressor 370 (not shown) boosts pressure of the second portion of overheads vapors from about 0.053 atm or 0.066 atm (i.e., about 40 or 50 mm Hg) to about 0.16 atm (i.e., about 120 mm Hg) to produce partially compressed wet VOC gases, and the second vacuum pump/compressor 380 (not shown) boosts pressure from 0.16 atm up to about 1 atm to give compressed wet gases;
an unfiltered ABE fermentation broth is essentially continuously fed from a enclosed volumetric space of the standard fermentor vessel (e.g. 211) to an enclosed volumetric space of the stripper vessel (e.g. 78 of FIG. 1 and 178 of FIG. 2) at a flow rate of from 7.6 liters per minute (Lpm) to 76 Lpm per 3790 liters of ABE fermentation broth disposed in the standard fermentor vessel (e.g., 211); or at a flow rate in the range of about 11 liters of fermentation broth per minute (L/min) to 38 L/min (i.e., about 3 gallons per minute (gal/min) to 10 gal/min) per 3790 liters (i.e., 1000 gallons) of fermentor vessel capacity;
when injecting a stripping gas from a stripping gas source 11 (not shown), the injecting is carried out at an adjustable flow rate of the stripping gas being injected to maintain a pressure in the enclosed volumetric space of the stripper vessel (e.g. 78 of FIG. 1 and 178 of FIG. 2) of from 0.066 atmospheres to 0.33 atmospheres;
an amount of stripping gas being injected is in the range of 1.7 kilogram stripping gas per liter (kg/L) stripper feed to 4.1 kg/L (i.e., 3 pounds per gallon (lb/gal) to 7 lb/gal); and
a vapor/liquid condenser 600 (not shown) is maintained at about −2° C. or higher, preferably at from about −1° C. to about 2° C.; and
a VSS unit (e.g., 10 of FIG. 1 or 100 of FIG. 2) in an ABE fermentation is operated at a pressure of 0.26 atm (i.e., 200 mm Hg).

For production of 1-butanol in a typical ABE fermentation broth using Clostridium beijerinckii as the organism, and a reboiled fermentor vessel (e.g., 222, not shown) one or more of the following operation parameters are preferred:
startup procedures and operation parameters for an ABE fermentation employing a reboiled fermentor vessel (e.g., 222, not shown) are the same as startup procedures and operation parameters for an ABE fermentation employing a standard fermentor vessel 211 (not shown) except as noted below; once concentration of total VOCs in the ABE fermentation broth in the reboiled fermentor vessel 222 has attained a level of from about 0.3 wt % to about 0.4 wt %, then pressure in the reboiled fermentor vessel 222 is gradually reduced until boiling of one or more VOCs begins;
once stripping of VOCs is started, a stripping rate is adjusted to maintain concentration of total VOCs in the ABE fermentation broth at or below about 1.2 wt %, and at or above about 0.5 wt %; more preferably at about 0.8 wt % total VOCs;
operation parameters for the VSS unit (e.g., 10 in FIG. 1 or 100 in FIG. 2) and vapor/liquid condenser 600 (not shown) are as described above;
for an ABE fermentation broth at 35° C. in the reboiled fermentor vessel 222, the pressure in the reboiled fermentor vessel 222 at which boiling begins will correspond to from about 0.059 atm to about 0.066 atm (i.e., about 45 mmHg to 50 mm Hg);
no nitrogen sweep or fermentation gas (e.g., CO₂) is recirculated through the reboiled fermentor vessel 222; and
a portion of partially-compressed wet VOC gases exiting a first vacuum pump/compressor 370 (not shown) may be fed through a heat exchanger (e.g., 214, not shown) in the reboiled fermentor vessel 222 to cool the partially compressed wet VOC gases.

Operation of the invention apparatus and process can be reliably demonstrated using simulation modeling. Generally, developing accurate simulation models of processes such as the invention process are well known and taught in Molecular Thermodynamics of Fluid-Phase Equilibria by J. M. Prausnitz, R. N. Lichtenthaler, and E. Gomez, 3^{rd} edition, Prentice-Hall, New York, 1999; and Analysis, Synthesis, and Design of Chemical Processes by R. Turton, R. C. Bailie, W. B. Whiting, and J. A. Shaeiwitz, 2^{nd} edition, Prentice-Hall, New York, 2002. Some embodiments of the invention apparatus comprise, among other units, a DWC unit 500 (not shown). A DWC unit 500 may be simulated, designed, and operated using techniques that are analogous to techniques known for simulation, design, and operation of standard distillation columns In using these known techniques in the present invention, the DWC unit 500 can be treated as an assembly of interconnected distillation columns disposed within a single shell or column, as described by Mutalib, Abdul, and Smith [“Operation and Control of Dividing Wall Distillation Columns Part 1: Degrees of Freedom and Dynamic Simulation,” Chem. Eng. Res. Des., vol. 76, pp. 308-318 (1998), and “Operation and Control of Dividing Wall Distillation Columns Part 2: Simulation and Pilot Plant Studies Using Temperature Control,” Chem. Eng. Res. Des., vol. 76, pp. 319-334 (1998)].

Some embodiments of the invention apparatus comprise, among other units, a PSA unit 400 (not shown). The PSA unit 400 is simulated using well-known methods. The PSA unit's operating cycle is based on the well-known Skarstrom cycle which utilizes two vessels or packed beds containing a suitable adsorbent. As described in Gas Separation by Adsorption Processes, Ralph T. Yang, supra, the Skarstrom operating cycle consists of (1) bed pressurization, (2) adsorption under pressure, (3) countercurrent blow down under reduced pressure, and (4) backpurge at reduced pressure. Each bed progresses through these 4 steps. While one bed is on line adsorbing water from the feed stream, the other bed is off line undergoing regeneration using a fraction of the dry organic product stream. Such a simple Skarstrom cycle is satisfactory for simulating a PSA unit 400 because only one component (water) of an organics liquid layer is strongly adsorbed.

When simulating an ABE fermentation process using a PSA unit 400 containing 3 angstrom molecular sieve adsorbent, 1-butanol, ethanol, and acetone adsorption can be neglected since molecules of butanol, ethanol, and acetone are much larger than the 3 A molecular sieve adsorbent pore sizes. Preferably, the PSA unit 400 operation is modeled assuming that gas phase vapors behaves ideally, that radial concentration profiles can be ignored, that bulk convection dominates diffusion so that the effective diffusivity of all components can be set to zero, and that only water is adsorbed by the adsorbent in the PSA unit 400. Adsorption isotherm approximations are made using the Langmuir isotherm, A=C1*C2 exp(b/T)*Ps/(1+C2*exp(b/T)*Ps), where C1, C2, and b are constants, T is temperature, Ps is saturation pressure of water, exp (x) is the exponential function e^{x}, and A is the amount of water adsorbed. These approximations allow for convenient simulation of a process and yield results that are sufficiently accurate to demonstrate the effectiveness of the PSA unit 400 operation. The resulting model equations are readily solved by using a partial differential equation solver such as FlexPDE by PDE Solutions, Inc., Spokane Valley, Wash., USA; or MATLAB® PDE toolbox by The MathWorks, Inc., Natick, Mass., USA. They may also be solved by using any ordinary differential equation solver by first discretizing in the axial z-dimension using the Method of Lines.

The invention is further illustrated by the following example.

### Example 1

Aspen Plus™ (trademark of AspenTech) software from AspenTech Engineering Suite 12.1 (the Program) is used to calculate process performance and identify preferred operating conditions and design specifications for a standard fermentor unit 200 (not shown) plus an agitation VSS unit 10. The Aspen flow sheet is shown in FIG. 3. The Aspen Plus™ simulation is constructed to utilize validated vapor-liquid equilibrium (VLE) and liquid-liquid equilibrium (LLE) models. The VLE and LLE models include acetone, butanol, ethanol, and water, as well as inert gas. The results of the simulation are summarized in Table 1. For these calculations, productivity of ABE fermentation is assumed to be 1.5 grams of 1-butanol produced per liter of fermentor vessel volume per hour, a typical value. The preferred operating and design conditions and the resulting performance are highlighted, assuming the VSS unit 10 can operate at 200 mm Hg without damaging the cells. The actual pressure limit can be readily determined through routine experiment.

TABLE 1
Results of the Aspen Simulation of ABE fermentation with a standard Fermentor unit 200 and agitation VSS unit 10.

| | | Stripper | | Ratio of Mass | | | | | | | | |
|---|---|---|---|---|---|---|---|---|---|---|---|---|
| | Stripper | Feed to | Stripper | Fraction BuOH in | | Mass | | | | | Stripper | |
| | Bottoms to | Fermentor | Feed to | Feed to Stripper | Mass | Fraction | | | Number of | | Temp. at | |
| Produc- | Stripper | Volume | Stripper | to Mass Fraction | Fraction | BuOH in | Strip- | Number of | Theoret- | Pres- | Stripper | Stripper |
| tivity | Feed | Ratio | Gas Mass | BuOH in Liquid | BuOH in | Liquid | ping | Transfer | ical | sure | Feed | Bottoms |
| g/L | Ratio | gal/min/ | Ratio | Leaving Stripper | Feed to | Leaving | Fac- | Units | Stages | mm | Stage | Temp. |
| broth/h | wt/wt | gal | wt/wt | Xin/Xout | Stripper | Stripper | tor | NTU | NTS | Hg | ° C. | ° C. |
| 1.5 | 0.9000 | 0.00995 | 11.95 | 1.94 | 0.004924 | 0.00254 | 0.73 | 3.4 | 4 | 200 | 40.0 | 49.0 |
| 1.5 | 0.9850 | 0.02380 | 85.67 | 1.22 | 0.005130 | 0.00420 | 0.19 | 1.6 | 4 | 200 | 45.6 | 62.9 |
| 1.5 | 0.9850 | 0.01067 | 38.45 | 1.73 | 0.005260 | 0.00304 | 0.94 | 3.9 | 4 | 200 | 46.9 | 60.1 |
| 1.5 | 0.9850 | 0.01350 | 23.21 | 1.49 | 0.005220 | 0.00351 | 0.48 | 2.7 | 4 | 200 | 42.5 | 55.5 |
| 1.5 | 0.9850 | 0.01500 | 19.50 | 1.42 | 0.005155 | 0.00364 | 0.47 | 2.7 | 4 | 200 | 39.5 | 52.7 |
| 1.5 | 0.9800 | 0.01250 | 13.70 | 1.61 | 0.004975 | 0.00304 | 0.59 | 3.0 | 4 | 200 | 39.0 | 48.8 |
| 1.5 | 0.9990 | 0.12720 | 538.9 | 1.02 | 0.004899 | 0.00525 | 0.03 | 0.4 | 4 | 200 | 40.8 | 65.1 |
| 1.5 | 0.9980 | 0.08580 | 199.4 | 1.04 | 0.005340 | 0.00479 | 0.07 | 0.8 | 4 | 200 | 40.2 | 64.4 |
| 1.5 | 0.9980 | 0.11510 | 133.7 | 1.00 | 0.004280 | 0.00416 | 0.08 | 0.9 | 4 | 200 | 36.5 | 63.7 |
| 1.5 | 0.9000 | 0.00534 | 8.89 | 90.1 | 0.004690 | 0.000052 | 2.39 | 6.0 | 4 | 200 | 53.9 | 57.0 |
| 1.5 | 0.9000 | 0.00534 | 4.69 | 56.8 | 0.004720 | 0.000083 | 2.56 | 6.2 | 4 | 200 | 47.2 | 50.3 |
| 1.5 | 0.9000 | 0.00530 | 2.19 | 24.7 | 0.004819 | 0.000195 | 2.56 | 6.2 | 4 | 200 | 37.5 | 38.6 |
| 1.5 | 0.9000 | 0.00534 | 1.28 | 12.0 | 0.005017 | 0.000420 | 2.51 | 6.1 | 4 | 200 | 29.8 | 28.3 |
| 1.5 | 0.9500 | 0.00590 | 5.34 | 4.6 | 0.005240 | 0.001128 | 1.32 | 4.6 | 4 | 200 | 39.3 | 43.0 |

The fermentor unit 200 simulated in Example 1 is given a gassed volume of 1012 cubic meters and working volume of 810 cubic meters and is determined to have a theoretical butanol production rate of 1200 kilograms of butanol per hour. For the simulation of Example 1, well-known process simulation methods described by J. M. Prausnitz, R. N. Lichtenthaler, and E. Gomez de Azevedo [Molecular Thermodynamics of Fluid-Phase Equilibria, 3rd Ed., Prentice-Hall, 1999] and R. Turton, R. C. Bailie, W. B. Whiting, and J. A. Shaeiwitz [Analysis, Synthesis, and Design of Chemical Processes, 2nd Ed., Prentice-Hall, 2002] are used with the Program, readily available literature validated VLE and LLE data, and physical properties provided by the Program to construct a simulation of an quaternary system (acetone, butanol, ethanol, and water) ABE fermentation process using the instant invention apparatus and process. All literature validated VLE and LLE data are obtained from The Dortmund Databank of physical properties, which is available from DDBST GmbH, Oldenburg, Germany (see, for example, Dortmund Databank set numbers [3], [11], [384], [388], [389], [392], [394], [552], [564], [565], [1134], [1464], [1593], [2121], [2338], [2349], [3262], [3861], [4550], [4551], [4802], [5616], [5778], [6681], [6696], [7349], [7824], [8092], [8209], [9570], [9571], [9572], [9576], [9579], [9580], [10491], [10582], [11767], [20749], [22417], and [23689]). The VLE and LLE models included acetone, butanol, ethanol, and water, as well as inert gas. Accurate representation of the phase equilibrium is developed using a non-random two liquid activity coefficient (NRTL) and a UNIQUAC activity coefficient (UNIQUAC) phase equilibrium models by regression of literature phase equilibrium data, including data sets for binary and ternary systems. Models are developed for dilute-solvent conditions present in an aqueous ABE fermentation broth, as well as for more concentrated VLE and LLE existing in downstream processing equipment including distillation units, vapor/liquid condensers 600, and liquid-liquid phase separators (e.g., decanters 650). NRTL model parameter values are listed in Table 2 and UNIQUAC model parameter values are listed in Table 3.

TABLE 2
NRTL Model Parameter Set

| Component i | WATER | BUTANOL | WATER | ACETONE | ACETONE | BUTANOL | Aspen |
|---|---|---|---|---|---|---|---|
| Component j | ACETONE | WATER | ETHANOL | BUTANOL | ETHANOL | ETHANOL | Parameter |
| Temperature units | K | K | K | K | K | K | Name |
| aij | 0 | −9.22757 | 3.4578 | 0 | −0.3471 | 0 | NRTL/1 |
| aji | 0 | 21.58982 | −0.8009 | 0 | −1.0787 | 0 | NRTL/1 |
| bij | 602.5584 | 715.4858 | −586.0809 | −43.1411 | 206.5973 | 8.4365 | NRTL/2 |
| bji | 317.5539 | −2476.49 | 246.18 | 299.2181 | 479.05 | 33.483 | NRTL/2 |
| cij | 0.5343 | 0.252074 | 0.3 | 0.3 | 0.3 | 0.3467 | NRTL/3 |
| dij | 0 | 2.54E−03 | 0 | 0 | 0 | 0 | NRTL/4 |
| eij | 0 | 0 | 0 | 0 | 0 | 0 | NRTL/5 |
| eji | 0 | 0 | 0 | 0 | 0 | 0 | NRTL/5 |
| fij | 0 | 0.022577 | 0 | 0 | 0 | 0 | NRTL/6 |
| fji | 0 | −0.03105 | 0 | 0 | 0 | 0 | NRTL/6 |

TABLE 3
UNIQUAC Model Parameter Set

| Component i | WATER | WATER | WATER | ACETONE | ACETONE | BUTANOL | Aspen |
|---|---|---|---|---|---|---|---|
| Component j | ACETONE | BUTANOL | ETHANOL | BUTANOL | ETHANOL | ETHANOL | Parameter |
| Temperature Units | ° K | ° K | ° K | ° K | ° K | ° K | Name |
| | | — | | — | | — | |
| aᵢ | −4.8338 | −5.1730362 | −2.4936 | −0.4425179 | −0.1179 | −4.72766773 | UNIQ/1 |
| aⱼ | 8.6051 | −5.56845496 | 2.0046 | 4.74898161 | 0.6983 | 0.538254347 | UNIQ/1 |
| bᵢ | 1612.196 | 932.602831 | 756.9477 | 221.962329 | −61.8807 | 1051.38 | UNIQ/2 |
| bⱼ | −3122.58 | 714.560217 | −728.971 | −1895.5968 | −234.671 | 0.466052255 | UNIQ/2 |
| cᵢ | 0 | 0 | 0 | 0 | 0 | 0 | UNIQ/3 |
| cⱼ | 0 | 0 | 0 | 0 | 0 | 0 | UNIQ/3 |
| dᵢ | 0 | 3.63E−03 | 0 | 0 | 0 | 0 | UNIQ/4 |
| dⱼ | 0 | 0.01090271 | 0 | 0 | 0 | 0 | UNIQ/4 |
| eᵢ | 0 | 0 | 0 | 0 | 0 | 0 | UNIQ/7 |
| eⱼ | 0 | 0 | 0 | 0 | 0 | 0 | UNIQ/7 |

Binary parameters for both NRTL and UNIQUAC models are regressed using the Data Regression System (DRS) of the Program with maximum-likelihood as the objective function. The program's Britt-Leucke algorithm is utilized with the Program's Deming initialization method. Initial guesses for the parameters are often provided to aid convergence. The initial guess is often obtained from VLE-LIT or VLE-IG data found in The Dortmund Databank of physical properties. Scaling factors also are used to roughly scale the parameters to the range [−1,1] to help convergence. It is determined that the NRTL model has limited LLE prediction capability for the quaternary system of Example 1.

Accordingly, NRTL parameters for butanol+water, acetone+butanol and ethanol+butanol systems are regressed using not only binary VLE and LLE data, but also ternary VLE and LLE data. However, the DRS has difficulty converging NRTL parameters, so the UNIQUAC model is used instead, which converges much more readily when using many different data sets. NRTL is used for systems that are nearly binary; i.e., systems that contain two predominant species that comprise at least 95% of the total mass of the system. For ternary and quaternary systems, the UNIQUAC model is used. Examples of a fit of resulting NRTL and UNIQUAC model data to Dortmund Databank Set [2121] literature data are graphically shown in FIG. 4. Referring to FIG. 4, experimental data (i.e., Dortmund Databank Set [2121] literature data) for a quaternary system comprising acetone, ethanol, butanol, and water are plotted using a solid-diamond symbol, experimental LLE data (i.e., Dortmund Databank Set [2121] literature data) for a ternary system comprising acetone, butanol, and water are plotted using an open-diamond symbol, calculated UNIQUAC data are plotted with a solid line, calculated NRTL data are plotted with a dotted line, and dashed lines are drawn between plotted data.

The invention has been described with reference to various specific and preferred embodiments and techniques. However, it should be understood that many variations and modifications may be made while remaining within the spirit and scope of the invention.

1. A fermentation apparatus comprising a fermentor unit and a vacuum side stripper (VSS) unit;
the fermentor unit comprising a fermentor vessel, the fermentor vessel having an exterior surface and an interior surface, the fermentor vessel surfaces being spaced apart from, and generally parallel to, each other so as to define an enclosed volumetric space, the fermentor vessel having defined therein at least two apertures, a first fluid aperture and a second fluid aperture, the at least two apertures being in fluid communication with the enclosed volumetric space;
the VSS unit comprising a stripper vessel, the stripper vessel having an exterior surface and an interior surface, the stripper vessel surfaces being spaced apart from, and generally parallel to, each other so as to define an enclosed volumetric space; the stripper vessel having disposed therein two or more side-by-side stripper compartments, each stripper compartment being separated from an adjacent stripper compartment by a vertical partition member; at least a bottom portion of each vertical partition member either having defined therein a liquid trafficking aperture or being spaced apart from a bottom portion of the stripper vessel so as to define a liquid trafficking conduit between the bottom portion of the partition member and the bottom portion of the stripper vessel, or a combination thereof; at least a top portion of each vertical partition member either having defined therein a vapor trafficking aperture or being spaced apart from a top portion of the stripper vessel so as to define a vapor trafficking conduit between the top portion of the partition member and the top portion of the stripper vessel, or a combination thereof; the stripper compartments being in sequential fluid communication with each other, adjacent stripper compartments being in fluid communication with each other via a liquid trafficking conduit, a liquid trafficking aperture, or a combination thereof, and via a vapor trafficking conduit, a vapor trafficking aperture, or a combination thereof; the stripper vessel having defined therein at least four apertures, a third fluid aperture, a gas aperture, a vapor aperture, and a first agitation aperture, each of the at least four apertures being in fluid communication with the enclosed volumetric space of the stripper vessel;
the first fluid aperture of the fermentor vessel being operatively connected to the third fluid aperture of the stripper vessel to establish fluid communication between the fermentor vessel and the stripper vessel.

2. The apparatus as in claim 1, the apparatus further comprising one or more additional components selected from the group consisting of: a blower; a first vacuum pump/compressor; a second vacuum pump/compressor; a vapor/liquid condenser; a decanter; a liquid-liquid extractor; a means for separating at least one volatile organic compound from water (means for separating); a dividing-wall distillation column (DWC) unit; an ultrafiltration membrane; and an membrane vessel, wherein the blower, first vacuum pump/compressor, second vacuum pump/compressor, vapor/liquid condenser, decanter, liquid-liquid extractor, means for separating, and DWC unit independently are in operative connection to, and fluid communication with the vapor aperture of the stripper vessel; the ultrafiltration membrane being disposed within the membrane vessel or the membrane vessel is absent and the ultrafiltration membrane being disposed within the fermentor vessel; the membrane vessel being in operative connection to, and fluid communication with, the first fluid aperture of fermentor vessel and the third fluid aperture of the stripper vessel; fluid communication being established between the fermentor vessel, stripper vessel and the one or more additional components.

3. The apparatus as in claim 2, the means for separating at least one volatile organic compound from water comprises a pressure-swing adsorption (PSA) unit.

4. The apparatus as in claim 3, wherein the one or more additional components comprise the PSA unit and the DWC unit, the vapor aperture of the stripper vessel being in sequential operative connection to, and fluid communication with, the PSA unit and DWC unit.

5. The apparatus as in claim 2, wherein the one or more additional components comprise the ultrafiltration membrane.

6. The apparatus as in claim 2, wherein the one or more additional components comprise the first vacuum pump/compressor, second vacuum pump/compressor, vapor/liquid condenser, decanter, PSA unit, and DWC unit, the vapor aperture of the stripper vessel being in sequential operative connection to, and fluid communication with, the first vacuum pump/compressor, second vacuum pump/compressor, vapor/liquid condenser, decanter, PSA unit, and DWC unit.

7. The apparatus as in claim 1, wherein the stripper vessel and fermentor vessel each further defining a return fluid aperture, the return fluid aperture of the stripper vessel being in operative connection to, and fluid communication with, the return fluid aperture of the fermentor vessel.

8. A process comprising the steps of: disposing a fermentation broth in the enclosed volumetric space of a fermentor vessel of an apparatus as in claim 1, the broth comprising water, a plurality of cells of an organism, and a nutrients feed; allowing the nutrients feed to be fermented by the organism to produce at least one volatile organic compound (VOC); and stripping the at least one VOC from the fermentation broth.

9. The process as in claim 8, wherein each of the at least one VOC has a molecular weight of less than 250 grams per mole.

10. The process as in claim 9, wherein each of the at least one VOC independently is selected from the group consisting of: HO—(C₁-C₈)alkyl; HO—(C₂-C₈)alkylene-O—(C₁-C₄)alkyl; (C₃-C₈)alkanone; HO—(C₃-C₈)alkanone; (C₁-C₈)alkyl-C(O)O—(C₁-C₄)alkyl; [oxo-(C₃-C₈)alkyl]-C(O)O—(C₁-C₄)alkyl; (C₀-C₆)alkylene-[C(O)O—(C₁-C₄)alkyl]₂; O—[(C₁-C₄)alkyl]₂; and [oxo-(C₂-C₄)alkyl]-O—(C₁-C₄)alkyl.

11. The process as in claim 8, wherein the organism is selected from the group consisting of: an Acetobacter, Alcaligenes, Arthrobacter, Bacillus, Brevibacterium, Candida, Clostridium, Corynebacterium, Enterococcus. Erwinia, Escherichia, Flavobacterium, Gluconobacter, Hansenula, Klebsiella, Lactobacillus, Methylobacterium, Micrococcus, Mycobacterium, Nocardia, Paenibacillus Pichia, Pseudomonas, Rhodococcus, Saccharomyces, Salmonella, Thermoanaerobacter, Xanthobacter, and Zymomonas.

12. The process as in claim 11, the process further comprising the step of feeding unfiltered fermentation broth from the enclosed volumetric space of the fermentor vessel to the enclosed volumetric space of the stripper vessel; wherein the organism is Clostridium acetobutylicum or Clostridium beijerinckii; the cells of the organism in the fermentation broth in the fermentor vessel are at a concentration of from 20 grams per liter (g/L) to 120 g/L; the time-averaged total concentration of the at least one VOC in the fermentation broth in the fermentor vessel is from 0.1 wt % to 2.0 wt; the unfiltered fermentation broth is essentially continuously fed from the enclosed volumetric space of the fermentor vessel to the enclosed volumetric space of the stripper vessel at a flow rate of from 7.6 liters per minute (Lpm) to 76 Lpm per 3790 liters of fermentation broth disposed in the fermentor vessel.
